(19) United States
(12) Patent Application Publication
(10) Pub. No.: US 2024/0109945 A1
(43) Pub. Date: Apr. 4, 2024

(54) REAGENTS FOR DETECTION OF N-GLYCOSYLATION

(71) Applicants: UNIVERSITY OF GEORGIA RESEARCH FOUNDATION, INC., Athens, GA (US); GLYCOSENSORS AND DIAGNOSTICS, LLC, Athens, GA (US)

(72) Inventors: Loretta Yang, San Diego, CA (US); Shengcheng Wu, Athens, GA (US); Robert J. Woods, Athens, GA (US); Christian Gerner-Smidt, Bogart, GA (US); Lu Meng, Watkinsville, GA (US); Shaza Abnouf, Athens, GA (US)

(21) Appl. No.: 18/241,459

(22) Filed: Sep. 1, 2023

Related U.S. Application Data
(60) Provisional application No. 63/403,657, filed on Sep. 2, 2022.

(51) Int. Cl. C07K 14/47; B01D 15/38; B01J 20/32

(52) U.S. Cl. CPC C07K 14/47; B01D 15/3804; B01J 20/3274

(57) ABSTRACT

An asparagine-linked glycan (N-glycan) detection reagent is provided that utilizes Fbs variants engineered from mouse Fbs proteins, wherein the Fbs variants bind specifically to N-glycosylated peptides and proteins and not to non-glycosylated peptides and proteins, including wherein the Fbs variants bind to N-glycosylated peptides and proteins and not to corresponding non-glycosylated versions of the same peptides and proteins. Compositions and methods for developing and using N-glycosylation-specific analytical, diagnostic, and therapeutic tools are provided.

### CONTINUING APPLICATION DATA

This application claims the benefit of U.S. Provisional Application Ser. No. 63/403,657, filed Sep. 2, 2022, which is incorporated by reference herein.

### GOVERNMENT FUNDING

This invention was made with government support under Grant No. NIH OD035390, awarded by National Institutes of Health. The Government has certain rights in the invention.

### SEQUENCE LISTING

This application contains a Sequence Listing electronically submitted to the United States Patent and Trademark Office via Patent Center as an XML file entitled “0235-000301US01.xml” having a size of 25 kilobytes and created on Sep. 1, 2023. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### BACKGROUND

Glycans play crucial roles in nearly every aspect of biological processes and their distinct properties make them appealing as disease biomarker targets. The array of glycans linked to proteins or peptides in a cell or organism, commonly referred to as the glycosylation profile or “glycome” of the cell or organism, is a dynamic property that can depend on many features including cellular localization, temporal state, and metabolic state. Changes in the glycome are known to be associated with various disease states such as cancer, and such changes are sometimes part of a disease mechanism, e.g., they may be exploited for adhesion by pathogens. Thus, variations in glycosylation can serve as disease biomarkers, and may alter the pharmacological properties of disease treatments.

Glycans commonly occur as N-linked glycans or O-linked glycans. N-linked glycans result from attachment of a glycan (oligosaccharide) to the amide nitrogen of an asparagine (Asn) residue of a protein or peptide, i.e., N-linked glycosylation of a protein or peptide. O-linked glycans result from attachment of a glycan (oligosaccharide) to the oxygen atom of serine (Ser) or threonine (Thr) residues of a protein or peptide, i.e., O-linked glycosylation of a protein or peptide. Due to their highly branched and variably linked nature, glycans can pose a challenge for detection, purification, and structural analysis.

Efforts to develop reagents with carbohydrate specificity and affinity sufficient to detect and/or purify N-glycosylated proteins have been reported. F-box proteins that function as the substrate recognition subunits of SKP1-CUL1-F-box protein (SCF) E3 ubiquitin ligase complexes, and in particular mammalian N-glycan-recognizing F-box proteins that are reported to commonly bind to the innermost position of N-glycans through a unique small hydrophobic pocket in their loops. (Yoshida et al., 2019, Front Physiol; 10:104) have been investigated as potential sources for developing such reagents. Cytosolic Fbs1 and Fbs2 (Yoshida et al., 2003, J Biol Chem; 278(4):43877-84), and endomembrane-bound Fbs3 (Yoshida et al., 2017, Proc Natl Acad Sci U S A; 114(32):8574-8579), each have features of potential interest for developing N-glycan-specific reagents. The Fbs1 component of the E3 ubiquitin-ligase complex has been investigated due to its ability to recognize glycans as part of its biological role in ubiquitylation, e.g., its ability to bind to the glycan core of N-glycoproteins, including core-fucosylated glycans, and its ability to recognize and bind to N-glycan motifs when N-linked glycoproteins misfold (see, Chen et al., 2017, Nature Comm, 8;15487). Efforts to develop reagents based on Fbs1 (a.k.a. Fbx2, FBXO2, NFB42) include a recombinant human Fbs1 derivative protein developed as a tool for enrichment of N-glycopeptides (Chen et al., 2017, Nature Comm. 8;15487; U.S. Pat. No. 10,080,787 B2), although reported Fbs1-based reagents appear to need high salt concentrations (2 M NaCl) for efficient binding.

It is recognized that N-glycan binding reagents could provide tools to help detect glyco-biomarkers and benefit the field of glycomics/glycoproteomics by enabling functions such as sample pre-enrichment, separation of N-glycosylated proteins from corresponding non-glycosylated versions of the same proteins and isolation of N-glycosylated proteins from a sample.

Given the promise of Fbs1 proteins for conversion into a binding reagent for N-glycans, there is a need for further research on variants of Fbs1 proteins, including Fbs1 variants from non-human sources.

### SUMMARY OF THE INVENTION

The present disclosure provides a binding reagent that specifically binds to N-glycosylated peptides and proteins and does not show substantial binding to non-glycosylated peptides and proteins, wherein the binding reagent comprises an amino acid sequence having at least 90% identity to amino acid residues 125 to 297 (the sugar binding domain) of the murine Fbs1 protein (SEQ ID NO: 9) and having an amino acid substitution at position 153 and/or 154 of SEQ ID NO:9. In some aspects, the binding reagent further comprises an amino acid sequence having at least 90% sequence identity to amino acid residues 96 to 124 (the linker domain) of the murine Fbs1 protein (SEQ ID NO: 9). In some aspects, the substitution at position 153 and/or 154 comprises a E153R and/or a E154R substitution.

In some aspects, the binding reagent of further comprises a N-terminal immobilization tag. In some aspects, the N-terminal immobilization tag comprises the amino acid sequence

| (SEQ ID NO: 19) | |
|---|---|
| | MGSSHHHHHHHHSSGENLYFQSHMAS. |

In some aspects, the binding reagent further comprises an amino acid substitution at position 177 and/or 178. In some aspects, the substitution at position 177 and/or 178 comprises a F177Y and/or a E178R substitution.

In some aspects, the binding reagent protein further comprises an amino acid substitution at position 158 and/or 159. In some aspects, the substitution at position 159 comprises a N159G substitution.

In some aspects, a binding reagent disclosed herein comprises at least 90%, at least 95%, or at least 99% sequence identity to SEQ ID NO: 6, SEQ ID NO: 7, SEQ ID NO: 8, SEQ ID NO: 16, SEQ ID NO: 17, or SEQ ID NO: 18, wherein the binding reagent includes an E153R and/or E154R substitution at position 153 and/or 154.

The present disclosure provides a composition comprising a binding reagent as described herein.

The present disclosure provides a conjugate comprising a first component comprising a binding reagent as described herein covalently linked to a second component.

The present disclosure provides an affinity matrix comprising a binding reagent as described herein. In some aspects, the affinity matrix is selected from the group consisting of a solid support, surface, a column, a resin, a bead, a particle, and a nanoparticle.

The present disclosure provides an isolated polynucleotide encoding a binding reagent as described herein.

The present disclosure provides a vector comprising a polynucleotide as described herein.

The present disclosure provides a host cell comprising a polynucleotide or vector as described herein.

The present disclosure provides a method for detecting an N-glycosylated protein in a sample, the method comprising contacting the sample with a binding reagent, composition, conjugate, or affinity matrix as described herein under conditions to allow binding of the binding reagent to the N-glycosylated protein and detecting the N-glycosylated protein bound to the binding reagent.

The present disclosure provides a method for enriching, isolating, or purifying an N-glycosylated protein, the method comprising contacting a biological or laboratory sample with a binding reagent, composition, conjugate, or affinity matrix as described herein under conditions to allow binding of the binding reagent to an N-glycosylated protein in the sample, and recovering the N-glycosylated protein bound to the binding reagent. In some aspects, the method further comprises separating the recovered N-glycosylated protein from the binding reagent.

As used herein, “isolated” refers to material removed from its original environment (e.g., the natural environment if it is naturally occurring), and thus is altered “by the hand of man” from its natural state.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

In several places throughout the application, guidance is provided through lists of examples, which examples can be used in various combinations. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

All headings throughout are for the convenience of the reader and should not be used to limit the meaning of the text that follows the heading, unless so specified.

### BRIEF DESCRIPTION OF DRAWINGS

FIGS. 1A and 1B show alignment of Fbs sequences. FIG. 1A shows the alignment of wild-type mouse (Mus musculus) mmFBXO2 sequence (SEQ ID NO: 9) with human (Homo sapiens) F-Box only protein 2 (hsFBXO2 (Chen et al., 2017, Nature Comm, 8; 15487), SEQ ID NO: 20) using EMBOSS Needle pairwise sequence alignment software. FIG. 1B shows a multiple sequence alignment of Fbs1 (mmFBXO2) (SEQ ID NO: 9), Fbs2 (mmFBXO6) (SEQ ID NO: 21), and Fbs3 (FBXO27) (SEQ ID NO: 22) using the CLUSTAL Omega (1.2.4) multiple sequence alignment tool (available on the worldwide web at, for example, clustal.org/omega/).

FIG. 2 shows biolayer interferometry (BLI) sensorgrams of variants Fbs1-1 and Fbs1-2 binding to biotinylated N-glycopeptides (A3Man, BRNBP) and a glycoprotein (bRNB) immobilized on SAX biosensors. Results for Fbs1-1 (left panel) and Fbs1-2 (right panel) binding are labelled as follows: BRNBP=SRN(M3N2)LTK; BRNAP=SRNLTK; A3Man=AVSHN(M3N2)ASLE; A3Pep=AVSHNASLE; bRNB=(biotinylated) RNase B; bRNA=(biotinylated) RNase A. Transient binding constants are based on the 2:1 curve fitting model.

FIG. 3 shows use of Western blot analysis of the binding of Fbs1 variants to glycoproteins and to non-glycosylated protein using variants Fbs1-1 and Fbs1-2 as probes. Lanes of each blot are labelled to show results for samples (1 μg each) of glycoproteins Asialofetuin, Fetuin, RNase B, Ovalbumin, Horse Radish Peroxidase, and IgG polyclonal antibody from rabbit, and a non-glycosylated protein RNase A, that were separated by SDS-PAGE, blotted on nitrocellulose membrane, and probed with Fbs1-1 (left blot) or Fbs1-2 (right blot). Comparison of the blots demonstrates that both Fbs1 variants (Fbs1-1 and Fbs1-2) bind to each glycoprotein (Asialofetuin, Fetuin, RNase B, Ovalbumin, Horse Radish Peroxidase, and IgG) and do not bind to RNase A which is the non-glycosylated form of RNase B. In addition, Fbs1-2 showed stronger binding to ovalbumin (high mannose and complex glycans) and HRP than Fbs1-1, indicating Fbs1-2 may be a more sensitive probe.

FIG. 4 shows results using ELISA for validation of the binding specificity of variant Fbs1-1, where plates coated with 10 μg/mL of each glycoprotein analyte were probed with 20 μg/mL Fbs1-1 in TBS buffer and detected with TMB substrate using an anti-poly-His HRP conjugate by measuring A₄₅₀. Fetuin is a heavily sialylated glycoprotein isolated from bovine serum; Asialofetuin is the desialylated form of Fetuin, IgG is a polyclonal immunoglobulin raised in rabbit, IgM an immunoglobulin raised in mouse, and IgY an immunoglobulin raised in chicken. The results showed that Fbs1-1 preferentially binds to desialylated Asialofetuin and IgM.

FIG. 5 shows results from glycan array screening of variant Fbs1-1 where an array of immobilized glycans as shown (structures from Gao et al., 2019, Cell Chem Biol; 26(4): 535-547) was incubated with 100 μg/mL biotinylated Fbs1-1 followed by 0.5 μg/mL of a streptavidin Cy5-635 conjugate. Fbs1-1 shows a preference for terminal GlcNAc, Gal, and Man N-glycans over terminally sialylated glycans.

FIGS. 6A-6C show results from a yeast display assay for validation of Fbs1-1 specificity, where yeast cells displaying Fbs1-1 on the cell surface were dual labeled with anti-c-Myc tag mouse mAb, followed by goat anti-mouse-Alexa Fluor 488 and either (FIG. 6A) the A3Man N-glycopeptide ligand precomplexed with streptavidin-Alex Fluor 633, (FIG. 6B) the A3Pep non-glycosylated peptide precomplexed with streptavidin-Alexa Fluor 633, or (FIG. 6C) the streptavidin-Alexa Fluor 633-only control. FACS results are shown in the upper row, and a simplified schematic of the interaction is shown in the bottom row. The results demonstrate that only N-glycopeptide binds to Fbs1-1 displayed on yeast surface (as shown in FIG. 6A for A3Man N-glycopeptide), but the corresponding non-glycosylated peptide does not bind to Fbs1-1 displayed on yeast surface (as shown in FIG. 6B for A3Pep non-glycosylated peptide precomplexed with streptavidin-Alexa Fluor 633), indicating that Fbs1-1 is amenable to directed evolution using yeast display technology.

FIG. 7 shows results using an Fbs1-1 affinity matrix for N-glycan-specific affinity chromatography for selective binding and sample enrichment. Fbs1-1 (5 mg) was chemically coupled to a 1-mL Sepharose column. The following analytes were evaluated: a glycoprotein, RNase B (middle panel); its non-glycosylated counterpart, RNase A (top panel); and a 1:1 mixture of RNase A and RNase B. Analytes were loaded on the Fbs1-1 column in TBS buffer, the column was washed, and bound protein was eluted with a low pH buffer (0.1M Glycine-HCl and 0.5M NaCl, pH 4.0). Flow-through fractions and elution peaks were collected, analyzed by SDS-PAGE, and compared with the original analyte (FIG. 7, right side, “SDS-PAGE”). FIG. 7 middle panel shows results using RNase B as a glycoprotein analyte (lane labelled “Input”) demonstrating that the Fbs1-1 affinity matrix bound the glycoprotein RNase B and the glycoprotein was collected in the elution fraction (lane labelled “Peak 2”), and RNase B was not detected in the unbound fraction (lane labelled “Flow-through”). FIG. 7 top panel shows results using RNase B as a non-glycosylated protein analyte (lane labelled “Input”) demonstrating that the Fbs1-1 affinity matrix did not bind the non-glycosylated RNase A, which passed through the column and was collected in the flow-through fraction (lane labelled “Flow-through”), and RNase A was not detected in the elution fraction (lane labelled “Peak 2”). FIG. 7 bottom panel shows results using a mixture of RNase A and RNase B as a mixture of glycoproteins and corresponding non-glycosylated protein (lane labelled “Input” in bottom panel of FIG. 7) demonstrating that the Fbs1-1 affinity matrix was able to selectively bind the glycoprotein RNase B (lane labelled “Peak 2”) while the non-glycosylated RNase A did not bind (lane labelled “Flow through”). Note that the peak indicated on the elution profile as “Peak 1” is an artifact that does not contain any protein.

FIG. 8. Amino acid sequences of variant Fbs1-1 (SEQ ID NO: 1), variant Fbs1-2 (SEQ ID NO: 2), variant Fbs1-3 (SEQ ID NO: 3), variant Fbs1-4 (SEQ ID NO: 4), variant Fbs1-5 (SEQ ID NO: 5), variant Fbs1-6 (SEQ ID NO: 6), variant Fbs1-7 (SEQ ID NO: 1), and variant Fbs1-8 (SEQ ID NO: 8).

FIGS. 9A and 9B. Modelling identifying residues 153 and 154 as targets for modification of Fbs1 for the Fbs1-6, Fbs1-7, and Fbs1-8 variants. As shown in FIG. 9A, GLU 40 and GLU 41 (E153 and E154, respectively on WT mouse Fbs1 (SEQ ID NO:9)) are both close to the Sia/Gal region in glycans with either α2-6 or α2-3 linkages. Their mutation to ARG may enhance affinity for these types of glycans. This is not based on MD simulation, and only uses reasonable single structures from GLYCAM.org with ARG rotamers selected from the Chimera rotamer library. As shown in FIG. 9B, GLU 41 (E154 on WT mouse Fbs1 ) is close to the Sia/Gal region in glycans with either α2-6 or α2-3 linkages. Its mutation to ARG may enhance affinity for these types of glycans. This is not based on MD simulation, and only uses reasonable single structures from GLYCAM.org. Common side chain rotamers of the ARG at 41 are shown in blue.

FIG. 10. Structure of ligands A3Pep, A3Man, and G2PB. Glycans are represented in Symbolic Nomenclature for Glycans (SNFG).

FIG. 11. Biolayer interferometry (BLI) sensorgrams of variants Fbs1-1, Fbs1-2, Fbs1-3, Fbs1-4, and Fbs1-5.

FIG. 12. Biolayer interferometry (BLI) sensorgrams of variants Fbs1-1, Fbs1-2, Fbs1-3, Fbs1-6, Fbs1-7, and Fbs1-8.

FIG. 13. Western blot analysis of the binding of variants Fbs1-1, Fbs1-3, Fbs1-6, Fbs1-7, and Fbs1-8. AF=Asialofetuin, F=Fetuin, Ribonuclease B=RNase B, OVA=Ovalbumin, and HRP=Horse Radish Peroxidase.

FIG. 14. Results from glycan array screening of variant Fbs1-7. Upper arrows indicate that binding to these N-glycans is higher than those shown in FIG. 5. However, the lower arrow indicates binding to sialylated N-glycans is lower than FIG. 5. This was unexpected because the E154R mutation was intended to enhance binding.

FIG. 15. Last frame from MD simulation of sialylated glycans with FBS.

### DETAILED DESCRIPTION OF ILLUSTRATIVE EMBODIMENTS

The present disclosure provides asparagine-linked glycan (N-glycan) detection reagents, also referred to herein as binding agents, that utilize the mouse Fbs proteins Fbs1, Fbs2, and Fbs3, and variants engineered from mouse these Fbs proteins, wherein the Fbs variants bind specifically to N-glycosylated peptides and proteins and do not show substantial binding to non-glycosylated peptides and proteins, including wherein the Fbs variants bind to N-glycosylated peptides and proteins and do not show substantial binding to corresponding non-glycosylated versions of the same peptides and proteins. The present disclosure provides Fbs variants suitable for use as N-glycan-specific analytical, diagnostic, and therapeutic agents. The present disclosure provides methods for developing and using Fbs variants as N-glycosylation-specific analytical, diagnostic, and therapeutic tools, and provides Fbs variants suitable for use in such methods. Fbs variants as provided herein are suitable for current use, and for further modification, in one or more N-glycosylation detection reagents. Fbs variants are provided herein for use in compositions and methods for developing and using N-glycosylation-specific analytical, diagnostic, and therapeutic tools.

The term “does not show substantial binding” to a non-glycosylated protein, as used herein, is understood to mean that the Fbs variant does not show detectable binding to a non-glycosylated protein target as would be expected in accordance with the non-limiting hypothesized mechanism whereby the Fbs variant binds to one or more structural features created by N-glycosylation of the protein target, and is further understood to mean that the Fbs variant may show very low but detectable binding to a non-glycosylated protein target under certain conditions in which non-specific binding or other association with the non-glycosylated protein target may occur.

Mouse (Mus musculus) Fbs1, also known as F-Box only protein 2, mmFBXO2, or mouseFbx2 (Mizushima et al., 2004, Nat Struct Mol Blot; 11(4):365-70; and Mizushima et al., 2007, Proc Natl Acad Sci USA; 104(14):5777-81), is a component of the E3 ubiquitin ligase that binds N-glycosylated peptides and proteins. The sequence of wild-type murine Fbs1 (SEQ ID NO: 9) is provided below and is annotated as follows:

Fbs1 (mmFBXO2)
(SEQ ID NO: 9)
MDGDGDPESVSHPEEASPEEQPEEAGAEASAEEEQLREAEEEEEAEAVEY
LAELPEPLLLRVLAELPATELVQACRLVCLRWKELVDGAPLWLLKCQQEG
LVPEGSADEERDHWQQFYFLSKRRRNLLRNPCGEEDLEGWSDVEHGGDGW
RVEELPGDNGVEFTQDDSVKKYFASSFEWCRKAQVIDLQAEGYWEELLDT
TQPAIVVKDWYSGRTDAGSLYELTVRLLSENEDVLAEFATGQVAVPEDGS
WMEISHTFIDYGPGVRFVRFEHGGQDSVYWKGWFGARVINSSVWVEP

PEST domain (residues 1-54 of mmFBXO2)
F-box domain (residues 55-95 of mmFBXO2)
Linker domain (residues 96-124 of mmFBXO2)
Sugar binding domain (residues 125-297 of mmFBXO2)

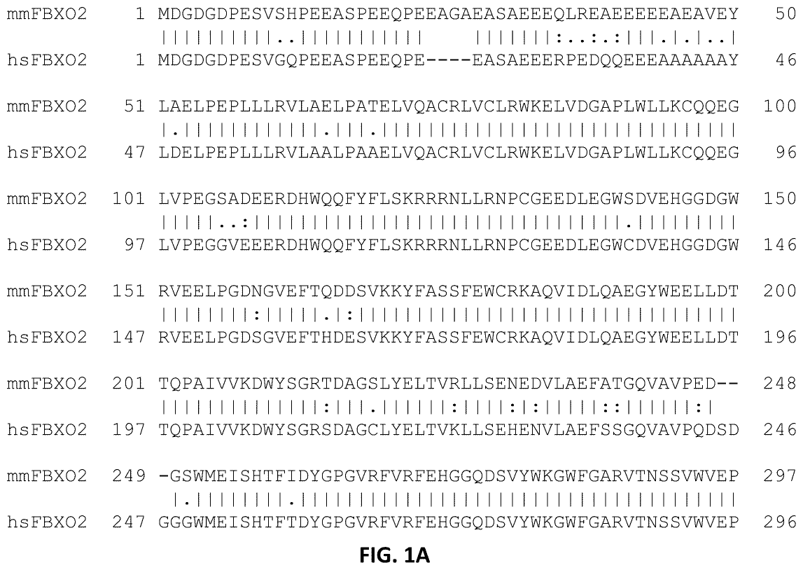

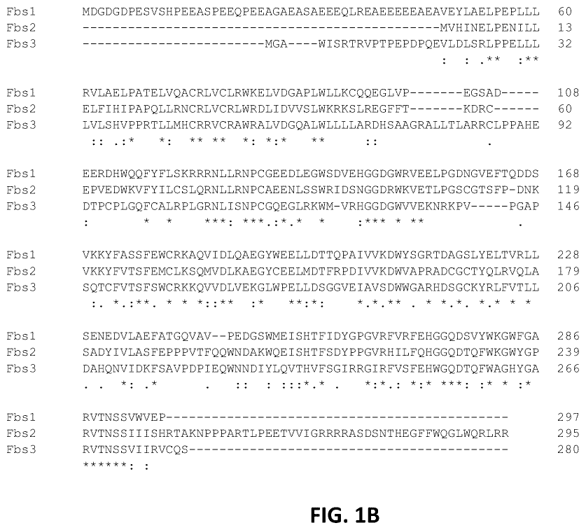

The native mouse F-box protein sequences for the Fbs2 (mmFBXO6 (SEQ ID NO: 21)) and Fbs3 (FBXO27 (SEQ ID NO: 22)) proteins are provided below. FIG. 1B shows a multiple sequence alignment of Fbs1, Fbs2, and Fbs3 using the CLUSTAL Omega (1.2.4) multiple sequence alignment tool (available on the worldwide at, for example, clustal.org/omega/).

Fbs2 (mmFBXO6)
(SEQ ID NO: 21)
MVHINELPENILLELFIHIPAPQLLRNCRLVCRLWRDLIDVVSLWKRKSL
REGFFTKDRCEPVEDWKVFYILCSLQRNLLRNPCAEENLSSWRIDSNGGD
RWKVETLPGSCGTSFPDNKVKKYFVTSFEMCLKSQMVDLKAEGYCEELMD
TFRPDIVVKDWVAPRADCGCTYQLRVQLASADYIVLASFEPPPVTFQQWN
DAKWQEISHTFSDYPPGVRHILFQHGGQDTQFWKGWYGPRVTNSSIIISH
RTAKNPPPARTLPEETVVIGRRRRASDSNTHEGFFWQGLWQRLRR
Fbs3 (FBXO27)
(SEQ ID NO: 22)
MGAWISRTRVPTPEPDPQEVLDLSRLPPELLLLVLSHVPPRTLLMHCRRV
CRAWRALVDGQALWLLLLARDHSAAGRALLTLARRCLPPAHEDTPCPLGQ
FCALRPLGRNLISNPCGQEGLRKWMVRHGGDGWVVEKNRKPVPGAPSQTC
FVTSFSWCRKKQVVDLVEKGLWPELLDSGGVEIAVSDWWGARHDSGCKYR
LFVTLLDAHQNVIDKFSAVPDPIEQWNNDIYLQVTHVFSGIRRGIRFVSF
EHWGQDTQFWAGHYGARVINSSVIIRVCQS

The present disclosure provides variants of a mouse Fbs protein, including, but not limited to variants of the mouse Fbs1, Fbs2, and Fbs3 proteins. Such a variant may have, for example, at least 90%, at least 95%, or at least 99% sequence identity to SEQ ID NO: 9, SEQ ID NO: 21, or SEQ ID NO:22, or a truncation thereof. Such a Fbs variant may have use as a binding reagent that specifically binds to N-glycosylated peptides and proteins and does not show substantial binding to non-glycosylated peptides and proteins. Such Fbs variants may be recombinantly expressed proteins.

A Fbs variant, including any of the Fbs variants described herein, may further have an N-terminal immobilization tag. An immobilization tag refers to a protein, nucleic acid, synthetic molecule, or other chemical capable of coupling with a binding reagent for purposes of immobilizing the N-glycan linked glycoprotein by affinity binding. Examples of immobilization tags are known in the art and include, but are not limited to, maltose-binding proteins (MBPs) (U.S. Pat. Nos. 5,643,758, 7,825,218, 7,883,867, and 8,623,615), SNAP-TAG (Kinderman et al., 2003, J Am Chem Soc; 125:7810-7811; New England Biolabs, Ipswich, MA), AGT (U.S. Pat. Nos. 7,939,284, 8,367,361, 7,799, 524, 7,888,090, 8,163,479, and 8,178,314), CLIP-TAG (U.S. Pat. Nos. 8,227,602 and 8,623,627), inteins (U.S. Pat. Nos. 5,496,714, 5,834,247, 6,521,425, 7,157,224, 6,849,428, 7,001,745, 6,858,775, and 7,271,256), chitin-binding proteins (U.S. Pat. Nos. 6,897,285, 7,060,465, 6,984,505, and 6,987.007), biotin, streptavidin, antibodies, Flag-tags, or a nuclear (NF)-κb p50 protein. The amino acid sequence of such an N-terminal immobilization tag is not found in the amino acid sequence of mammalian Fbs protein.

In one aspect, an N-terminal immobilization tag is a polyhistidine tag (also known as 8xHis) having the amino acid sequence MGSSHHHHHHHHSSGENLYFQSHMAS (SEQ ID NO: 19). The ENLYQS sequence of this tag is the TEV protease cleavage sequence, cleaving between the Q and S.

In some aspects, a Fbs variant includes a truncation of a mouse Fbs1 protein (SEQ ID NO: 9), a Fbs2 protein (SEQ ID NO: 21), or a Fbs3 protein (SEQ ID NO: 22), including, for example, a truncation that includes the sugar binding domain (SBD) (residues 125 to 297 of mmFBXO2). Such a truncation including the sugar binding domain may also include a portion of the linker domain (residues 96 to 124 of mmFBXO2).

One example of such a truncation is the Fbs1-1 variant, a variant of the mouse Fbs1 protein having amino acid residues 96 to 124 (the linker domain) and amino acid residues 125 to 297 (the sugar binding domain) of the murine Fbs1 protein (SEQ ID NO: 9).

The amino acid sequence of a Fbs1-1 variant without a N-terminal immobilization tag is:

(SEQ ID NO: 11)
QQEGLVPEGSADEERDHWQQFYFLSKRRRNLLRNPCGEEDLEGWSDVEHG
GDGWRVEELPGDNGVEFTQDDSVKKYFASSFEWCRKAQVIDLQAEGYWEE
LLDTTQPAIVVKDWYSGRTDAGSLYELTVRLLSENEDVLAEFATGQVAVP
EDGSWMEISHTFIDYGPGVRFVRFEHGGQDSVYWKGWFGARVINSSVWVE
P.

The Fbs1-1 variant with a N-terminal immobilization tag has the following sequence:

(SEQ ID NO: 1)
MGSSHHHHHHHHSSGENLYFQSHMASQQEGLVPEGSADEERDHWQQFYFL
SKRRRNLLRNPCGEEDLEGWSDVEHGGDGWRVEELPGDNGVEFTQDDSVK
KYFASSFEWCRKAQVIDLQAEGYWEELLDTTQPAIVVKDWYSGRTDAGSL
YELTVRLLSENEDVLAEFATGQVAVPEDGSWMEISHTFIDYGPGVRFVRF
EHGGQDSVYWKGWFGARVTNSSVWVEP.

A truncated Fbs1-1 variant may have an amino acid sequence having at least 90%, at least 95%, or at least 99% identity to amino acid residues 125 to 297 (the sugar binding domain) of the murine Fbs1 protein (SEQ ID NO: 9). A truncated Fbs1-1 variant may have one, two, three, four, five, or more amino acid differences form the amino acid sequence of residues 125 to 297 of the murine Fbs1 protein (SEQ ID NO: 9). A truncated Fbs1-1 variant may have the amino acid sequence of amino acid residues 125 to 297 (the sugar binding domain) of the murine Fbs1 protein (SEQ ID NO: 9).

A truncated Fbs1-1 variant may have an amino acid sequence having at least 90%, at least 95%, or at least 99% identity to SEQ ID NO: 1 or SEQ ID NO:11. A truncated Fbs1-1 variant may have the amino acid sequence SEQ ID NO: 1 or SEQ ID NO:11 with one, two, three, four, five, or more amino acid changes. A truncated Fbs1-1 variant may have the amino acid sequence SEQ ID NO: 1 or SEQ ID NO:11. A truncated Fbs1-1 variant of the present disclosure may bind specifically to N-glycosylated peptides and proteins and not to non-glycosylated peptides and proteins. A Fbs1-1 variant may show specific binding to N-glycans that can include, but is not limited to, tri- and tetra-antennary structures, and those that are α1,6 core fucosylated. In some embodiments, a Fbs1-1 as provided herein has a portion of the amino acid sequence set forth above sufficient to achieve the specific N-glycan binding activity of Fbs1-1.

In some aspects, a Fbs1 variant is a Fbs1-2 variant, a variant of the Fbs1-1 truncated protein with further amino acid modifications. A Fbs1-2 variant is the Fbs1-1 variant in which the amino acids at positions equivalent to positions 177 and 178 of the native murine Fbs1 protein (SEQ ID NO: 9) are modified, including, but not limited to, embodiments with F177Y and/or E178R substitutions (underlined in the sequences below).

The Fbs1-2 variant without a N-terminal immobilization tag has the following sequence:

(SEQ ID NO: 12)
QQEGLVPEGSADEERDHWQQFYFLSKRRRNLLRNPCGEEDLEGWSDVEHG
GDGWRVEELPGDNGVEFTQDDSVKKYFASSYRWCRKAQVIDLQAEGYWEE
LLDTTQPAIVVKDWYSGRTDAGSLYELTVRLLSENEDVLAEFATGQVAVP
EDGSWMEISHTFIDYGPGVRFVRFEHGGQDSVYWKGWFGARVINSSVWVE
P.

The Fbs1-2 variant with a N-terminal immobilization tag has the following sequence:

(SEQ ID NO: 2)
MGSSHHHHHHHHSSGENLYFQSHMASQQEGLVPEGSADEERDHWQQFYFL
SKRRRNLLRNPCGEEDLEGWSDVEHGGDGWRVEELPGDNGVEFTQDDSVK
KYFASSYRWCRKAQVIDLQAEGYWEELLDTTQPAIVVKDWYSGRTDAGSL
YELTVRLLSENEDVLAEFATGQVAVPEDGSWMEISHTFIDYGPGVRFVRF
EHGGQDSVYWKGWFGARVINSSVWVEP.

A Fbs1-2 variant may have an amino acid sequence having at least 90%, at least 95%, or at least 99% identity to SEQ ID NO: 2 or SEQ ID NO:12. A Fbs1-2 variant may have the amino acid sequence SEQ ID NO: 2 or SEQ ID NO:12 with one, two, three, four, five, or more amino acid changes. A Fbs1-2 variant may have the amino acid sequence SEQ ID NO: 2 or SEQ ID NO:12. A Fbs1-2 variant of the present disclosure may bind specifically to N-glycosylated peptides and proteins and not to non-glycosylated peptides and proteins. A Fbs1-2 variant may show specific binding to N-glycans that can include, but is not limited to, tri- and tetra-antennary structures, and those that are α1,6 core fucosylated. In some embodiments, a Fbs1-2 as provided herein has a portion of the amino acid sequence set forth above sufficient to achieve the specific N-glycan binding activity of Fbs1-2.

In some aspects, a Fbs1 variant is a Fbs1-3 variant, a variant in which an additional mutation is added to the Fbs1-2 variant. In addition to the modification of amino acids at positions 177 and 178 from FE to YR of the Fbs1-2 variant, the amino acid at a position equivalent to position 159 of the native murine Fbs1 protein (SEQ ID NO: 9) is modified, including, but not limited to, embodiments with a N159G modification. These modifications are underlined in the sequences below. In some embodiments, the amino acid at a position equivalent to position 158 of the native murine Fbs1 protein (SEQ ID NO: 9) is randomized.

The Fbs1-3 variant without a N-terminal immobilization tag has the following sequence:

(SEQ ID NO: 13)
QQEGLVPEGSADEERDHWQQFYFLSKRRRNLLRNPCGEEDLEGWSDVEHG
GDGWRVEELPGDGGVEFTQDDSVKKYFASSYRWCRKAQVIDLQAEGYWEE
LLDTTQPAIVVKDWYSGRTDAGSLYELTVRLLSENEDVLAEFATGQVAVP
EDGSWMEISHTFIDYGPGVRFVRFEHGGQDSVYWKGWFGARVINSSVWVE
P.

The Fbs1-3 variant with a N-terminal immobilization tag has the following sequence:

(SEQ ID NO: 3)
MGSSHHHHHHHHSSGENLYFQSHMASQQEGLVPEGSADEERDHWQQFYFL
SKRRRNLLRNPCGEEDLEGWSDVEHGGDGWRVEELPGDGGVEFTQDDSVK
KYFASSYRWCRKAQVIDLQAEGYWEELLDTTQPAIVVKDWYSGRTDAGSL
YELTVRLLSENEDVLAEFATGQVAVPEDGSWMEISHTFIDYGPGVRFVRF
EHGGQDSVYWKGWFGARVTNSSVWVEP.

A Fbs1-3 variant may have an amino acid sequence having at least 90%, at least 95%, or at least 99% identity to SEQ ID NO: 3 or SEQ ID NO:13. A Fbs1-3 variant may have the amino acid sequence SEQ ID NO: 3 or SEQ ID NO:13 with one, two, three, four, five, or more amino acid changes. A Fbs1-3 variant may have the amino acid sequence SEQ ID NO: 2 or SEQ ID NO:12. A Fbs1-3 variant of the present disclosure may bind specifically to N-glycosylated peptides and proteins and not to non-glycosylated peptides and proteins. A Fbs1-3 variant may show specific binding to N-glycans that can include, but is not limited to, tri- and tetra-antennary structures, and those that are α1,6 core fucosylated. In some embodiments, a Fbs1-3 as provided herein has a portion of the amino acid sequence set forth above sufficient to achieve the specific N-glycan binding activity of Fbs1-3.

In some aspects, a Fbs1 variant is a Fbs1-4 variant or a Fbs1-5 variant, variants of the Fbs1-1 truncated protein with further amino acid modifications.

A Fbs1-4 variant is a Fbs1-1 variant in which the amino acid at a position equivalent to position 214 of the native murine Fbs1 protein (SEQ ID NO: 9) is modified, including, but not limited to, embodiments with a R214A substitution. These modifications are underlined in the sequences below.

The Fbs1-4 variant without a N-terminal immobilization tag has the following sequence:

(SEQ ID NO: 14)
QQEGLVPEGSADEERDHWQQFYFLSKRRRNLLRNPCGEEDLEGWSDVEHG
GDGWRVEELPGDNGVEFTQDDSVKKYFASSFEWCRKAQVIDLQAEGYWEE
LLDTTQPAIVVKDWYSGATDAGSLYELTVRLLSENEDVLAEFATGQVAVP
EDGSWMEISHTFIDYGPGVRFVRFEHGGQDSVYWKGWFGARVTNSSVWVE
P.

The Fbs1-4 variant with a N-terminal immobilization tag has the following sequence:

(SEQ ID NO: 4)
MGSSHHHHHHHHSSGENLYFQSHMASQQEGLVPEGSADEERDHWQQFYFL
SKRRRNLLRNPCGEEDLEGWSDVEHGGDGWRVEELPGDNGVEFTQDDSVK
KYFASSFEWCRKAQVIDLQAEGYWEELLDTTQPAIVVKDWYSGATDAGSL
YELTVRLLSENEDVLAEFATGQVAVPEDGSWMEISHTFIDYGPGVRFVRF
EHGGQDSVYWKGWFGARVTNSSVWVEP.

A Fbs1-4 variant may have an amino acid sequence having at least 90%, at least 95%, or at least 99% identity to SEQ ID NO: 4 or SEQ ID NO:14. A Fbs1-4 variant may have the amino acid sequence SEQ ID NO: 4 or SEQ ID NO:14 with one, two, three, four, five, or more amino acid changes. A Fbs1-4 variant may have the amino acid sequence SEQ ID NO: 4 or SEQ ID NO:14. A Fbs1-4 variant of the present disclosure may bind specifically to N-glycosylated peptides and proteins and not to non-glycosylated peptides and proteins. A Fbs1-4 variant may show specific binding to N-glycans that can include, but is not limited to, tri- and tetra-antennary structures, and those that are α1,6 core fucosylated. In some embodiments, a Fbs1-4 as provided herein has a portion of the amino acid sequence set forth above sufficient to achieve the specific N-glycan binding activity of Fbs1-4.

A Fbs1-5 variant is a Fbs1-1 variant in which the amino acid at a position equivalent to position 216 of the native murine Fbs1 protein (SEQ ID NO: 9) is modified, including, but not limited to, embodiments with a D216A substitution. These modifications are underlined in the sequences below.

The Fbs1-5 variant without a N-terminal immobilization tag has the following sequence:

(SEQ ID NO: 15)
QQEGLVPEGSADEERDHWQQFYFLSKRRRNLLRNPCGEEDLEGWSDVEHG
GDGWRVEELPGDNGVEFTQDDSVKKYFASSFEWCRKAQVIDLQAEGYWEE
LLDTTQPAIVVKDWYSGRTAAGSLYELTVRLLSENEDVLAEFATGQVAVP
EDGSWMEISHTFIDYGPGVRFVRFEHGGQDSVYWKGWFGARVTNSSVWVE
P.

The Fbs1-5 variant with a N-terminal immobilization tag has the following sequence:

(SEQ ID NO: 5)
MGSSHHHHHHHHSSGENLYFQSHMASQQEGLVPEGSADEERDHWQQFYFL
SKRRRNLLRNPCGEEDLEGWSDVEHGGDGWRVEELPGDNGVEFTQDDSVK
KYFASSFEWCRKAQVIDLQAEGYWEELLDTTQPAIVVKDWYSGRTAAGSL
YELTVRLLSENEDVLAEFATGQVAVPEDGSWMEISHTFIDYGPGVRFVRF
EHGGQDSVYWKGWFGARVTNSSVWVEP.

A Fbs1-5 variant may have an amino acid sequence having at least 90%, at least 95%, or at least 99% identity to SEQ ID NO: 5 or SEQ ID NO:15. A Fbs1-5 variant may have the amino acid sequence SEQ ID NO: 5 or SEQ ID NO:15 with one, two, three, four, five, or more amino acid changes. A Fbs1-5 variant may have the amino acid sequence SEQ ID NO: 5 or SEQ ID NO:15. A Fbs1-5 variant of the present disclosure may bind specifically to N-glycosylated peptides and proteins and not to non-glycosylated peptides and proteins. A Fbs1-5 variant may show specific binding to N-glycans that can include, but is not limited to, tri- and tetra-antennary structures, and those that are α1,6 core fucosylated. In some embodiments, a Fbs1-5 as provided herein has a portion of the amino acid sequence set forth above sufficient to achieve the specific N-glycan binding activity of Fbs1-5.

In some aspects, a Fbs1 variant is a Fbs1-1 variant with substitution at positions equivalent to positions 153 and/or 154 of the native murine Fbs1 protein (SEQ ID NO: 9), including, but not limited to, embodiments with a E153R and/or a E154R substitution. For example, such Fbs1 variants include a Fbs1-6 variant, a Fbs1-7 variant, or a Fbs1-8 variant, variants of the Fbs1-3 variant with further amino acid modifications. For the Fbs1-6, Fbs1-7, and Fbs1-8 variants, additional substitutions are introduced into the Fbs1-3 variant at positions equivalent to positions 153 and/or 154 of the native murine Fbs1 protein (SEQ ID NO: 9), including, but not limited to, embodiments with a E153R and/or a E154R substitution. Thus, in addition to the FE to YR substitutions at amino acids positions 177 and 178 and the N to G substitution at position 159, the Fbs1-6, Fbs1-7, and Fbs1-8 variants have additional substitutions at positions 153 and/or 154. Specifically, the Fbs1-6 variant has an E to R substitution at position 153, the Fbs1-7 variant has an E to R substitution at position 154, and the Fbs1-8 variant has the E to R substitution at both residues 153 and 154.

The Fbs1-6 variant without a N-terminal immobilization tag has the following sequence:

(SEQ ID NO: 16)
QQEGLVPEGSADEERDHWQQFYFLSKRRRNLLRNPCGEEDLEGWSDVEHG
GDGWRVRELPGDGGVEFTQDDSVKKYFASSYRWCRKAQVIDLQAEGYWEE
LLDTTQPAIVVKDWYSGRTDAGSLYELTVRLLSENEDVLAEFATGQVAVP
EDGSWMEISHTFIDYGPGVRFVRFEHGGQDSVYWKGWFGARVTNSSVWVE
P.

The Fbs1-6 variant with a N-terminal immobilization tag has the following sequence:

(SEQ ID NO: 6)
MGSSHHHHHHHHSSGENLYFQSHMASQQEGLVPEGSADEERDHWQQFYFL
SKRRRNLLRNPCGEEDLEGWSDVEHGGDGWRVRELPGDGGVEFTQDDSVK
KYFASSYRWCRKAQVIDLQAEGYWEELLDTTQPAIVVKDWYSGRTDAGSL
YELTVRLLSENEDVLAEFATGQVAVPEDGSWMEISHTFIDYGPGVRFVRF
EHGGQDSVYWKGWFGARVTNSSVWVEP.

A Fbs1-6 variant may have an amino acid sequence having at least 90%, at least 95%, or at least 99% identity to SEQ ID NO: 6 or SEQ ID NO:16. A Fbs1-6 variant may have the amino acid sequence SEQ ID NO: 6 or SEQ ID NO:16 with one, two, three, four, five, or more amino acid changes. A Fbs1-6 variant may have the amino acid sequence SEQ ID NO: 6 or SEQ ID NO:16. A Fbs1-6 variant of the present disclosure may bind specifically to N-glycosylated peptides and proteins and not to non-glycosylated peptides and proteins. A Fbs1-6 variant may show specific binding to N-glycans that can include, but is not limited to, tri- and tetra-antennary structures, and those that are α1,6 core fucosylated. In some embodiments, a Fbs1-6 as provided herein has a portion of the amino acid sequence set forth above sufficient to achieve the specific N-glycan binding activity of Fbs1-6.

The Fbs1-7 variant without a N-terminal immobilization tag has the following sequence:

(SEQ ID NO: 17)
QQEGLVPEGSADEERDHWQQFYFLSKRRRNLLRNPCGEEDLEGWSDVEHG
GDGWRVERLPGDGGVEFTQDDSVKKYFASSYRWCRKAQVIDLQAEGYWEE
LLDTTQPAIVVKDWYSGRTDAGSLYELTVRLLSENEDVLAEFATGQVAVP
EDGSWMEISHTFIDYGPGVRFVRFEHGGQDSVYWKGWFGARVTNSSVWVE
P.

The Fbs1-7 variant with a N-terminal immobilization tag has the following sequence:

(SEQ ID NO: 7)
MGSSHHHHHHHHSSGENLYFQSHMASQQEGLVPEGSADEERDHWQQFYFL
SKRRRNLLRNPCGEEDLEGWSDVEHGGDGWRVERLPGDGGVEFTQDDSVK
KYFASSYRWCRKAQVIDLQAEGYWEELLDTTQPAIVVKDWYSGRTDAGSL
YELTVRLLSENEDVLAEFATGQVAVPEDGSWMEISHTFIDYGPGVRFVRF
EHGGQDSVYWKGWFGARVTNSSVWVEP.

A Fbs1-7 variant may have an amino acid sequence having at least 90%, at least 95%, or at least 99% identity to SEQ ID NO: 7 or SEQ ID NO:17. A Fbs1-7 variant may have the amino acid sequence SEQ ID NO: 7 or SEQ ID NO:17 with one, two, three, four, five, or more amino acid changes. A Fbs1-7 variant may have the amino acid sequence SEQ ID NO: 7 or SEQ ID NO:17. A Fbs1-7 variant of the present disclosure may bind specifically to N-glycosylated peptides and proteins and not to non-glycosylated peptides and proteins. A Fbs1-7 variant may show specific binding to N-glycans that can include, but is not limited to, tri- and tetra-antennary structures, and those that are α1,6 core fucosylated. In some embodiments, a Fbs1-7 as provided herein has a portion of the amino acid sequence set forth above sufficient to achieve the specific N-glycan binding activity of Fbs1-7.

The Fbs1-8 variant without a N-terminal immobilization tag has the following sequence:

(SEQ ID NO: 18)
QQEGLVPEGSADEERDHWQQFYFLSKRRRNLLRNPCGEEDLEGWSDVEHG
GDGWRVRRLPGDGGVEFTQDDSVKKYFASSYRWCRKAQVIDLQAEGYWEE
LLDTTQPAIVVKDWYSGRTDAGSLYELTVRLLSENEDVLAEFATGQVAVP
EDGSWMEISHTFIDYGPGVRFVRFEHGGQDSVYWKGWFGARVTNSSVWVE
P.

The Fbs1-8 variant with a N-terminal immobilization tag has the following sequence:

(SEQ ID NO: 8)
MGSSHHHHHHHHSSGENLYFQSHMASQQEGLVPEGSADEERDHWQQFYFL
SKRRRNLLRNPCGEEDLEGWSDVEHGGDGWRVRRLPGDGGVEFTQDDSVK
KYFASSYRWCRKAQVIDLQAEGYWEELLDTTQPAIVVKDWYSGRTDAGSL
YELTVRLLSENEDVLAEFATGQVAVPEDGSWMEISHTFIDYGPGVRFVRF
EHGGQDSVYWKGWFGARVTNSSVWVEP.

A Fbs1-8 variant may have an amino acid sequence having at least 90%, at least 95%, or at least 99% identity to SEQ ID NO: 8 or SEQ ID NO:18. A Fbs1-8 variant may have the amino acid sequence SEQ ID NO: 8 or SEQ ID NO:18 with one, two, three, four, five, or more amino acid changes. A Fbs1-8 variant may have the amino acid sequence SEQ ID NO: 8 or SEQ ID NO:18. A Fbs1-8 variant of the present disclosure may bind specifically to N-glycosylated peptides and proteins and not to non-glycosylated peptides and proteins. A Fbs1-8 variant may show specific binding to N-glycans that can include, but is not limited to, tri- and tetra-antennary structures, and those that are α1,6 core fucosylated. In some embodiments, a Fbs1-8 as provided herein has a portion of the amino acid sequence set forth above sufficient to achieve the specific N-glycan binding activity of Fbs1-8.

In some embodiments of a FBs1-6, Fbs1-7, or Fbs1-8 variant, rather than a E153R and/or a E154R substitution at positions equivalent to positions 153 and/or 154 of the native murine Fbs1 protein (SEQ ID NO: 9), the E(153) residue and/or the E(154) position may be substituted with a positively-charged residue (such as Arg or Lys) or a polar neutral residue (such as Gln, Asn, or His).

In further embodiments, a Fbs1 variant described above may further include a random substitution at a position equivalent to position 158 of the native murine Fbs1 protein (SEQ ID NO: 9).

In some embodiments a Fbs1 variant includes the amino acid sequence:

(SEQ ID NO: 10)
NLLRNPCGEEDLEGWSDVEHGGDGWRVXXLPGXXGVEFTQDDSVKKYFAS
SXXWCRKAQVIDLQAEGYWEELLDTTQPAIVVKDWYSGXTXAGSLYELTV
RLLSENEDVLAEFATGQVAVPEDGSWMEISHTFIDYGPGVRFVRFEHGGQ
DSVYWKGWFGARVTNSSVWVEP,

wherein any or all X residues are any amino acid.

In accordance with another aspect, the present disclosure provides a Fbs variant with binding specificity and/or binding kinetics and/or binding affinity that can be addressed by methods such as but not limited to targeted modifications, computational modeling, and directed evolution to develop and/or optimize the Fbs variant for use as a N-glycan-specific reagent with desired properties for use in one or more applications.

Fbs variants as provided herein have potential utility as current or future use as general N-glycan affinity reagents, also known as pan-specific N-glycan-binding reagents. Fbs1 variants as provided herein exhibit desirable properties for a pan-specific N-glycan-binding reagents from mouse Fbs1 (FBXO2) for the detection of N-linked glycans. Examples and Figures included herein have demonstrated that Fbs variants as provided here can be used for glycan enrichment by separating N-glycans from non-glycosylated components.

Further engineering of Fbs variants as provided herein, in particular of Fbs1 variants Fbs1-1, Fbs1-2, Fbs1-3, Fbs1-4, Fbs1-5, Fbs1-6, Fbs1-7, and Fbs1-8, or similar variants engineered of mouse Fbs2 and/or mouse Fbs3, can enhance desirable properties and/or add functionalities to support use of Fbs variants outside of their original function. An approach to further engineering of Fbs variants can use computational structural modeling to guide the design of clonal libraries for expression in yeast followed by affinity screening. An affinity matrix employing an Fbs variant may be used, for example, for enrichment of glycoproteins from a complex sample such as serum, biologic or cell lysate and for the enrichment of N-glycopeptides prior to glycosylation site-mapping by mass spectrometry. The performance of the Fbs variants can be compared to known reagents such as the lectins Concanavalin A (Con A) and Phaseolus vulgaris leucoagglutinin (PHA-L).

Additional Fbs variants as provided herein can be cloned, expressed (e.g., recombinantly produced in bacteria), and purified to characterize their affinity and specificity. One non-limiting exemplary approach will use a structure-based directed-evolution approach as described in U.S. Pat. No. 9,926,612, herein incorporated by reference. This approach enables binding thermodynamics, kinetics, and specificities of the reagents to be tuned to meet specific requirements begin by using the coordinates for Fbs1 to perform to perform simulations of the molecular dynamics (MD simulations) of the co-complex with a variety of N-linked glycans and glycopeptides based on alignment with the co-crystal structure for Fbs1 and the core of N-glycans, chitobiose (G1cNAc2), e.g., using the GLYCAM/AMBER force field. Initial structures for the Fbs1-glycan complexes can be generated by superimposing structures of the relevant glycans (generated using GLYCAM-Web, see the worldwide web at glycam.org) onto the coordinates of GlcNAc2. This approach can systematically validate the co-complex structures as the interaction interface increases in the binding pocket consistent with experimental applications. The data from the MD simulations can be employed to identify the optimal subset of residues that will be mutated and screened for high affinity clones that bind to N-glycans.

Selected optimized Fbs clones (selected for specificity, affinity, stability, etc.) can be paired with appropriate applications, i.e., affinity enrichment, Western blotting, immunohistochemistry, immunoprecipitation, and flow cytometry, in particular development of affinity matrices for N-glycan and glycoprotein enrichment. Further steps may include scaling up production capacity, and optimizing expression and purification protocols, comparison of engineered Fbs pan-specific detection reagent candidates with commonly used lectins for N-glycan enrichment. Standard operating procedures (SOPs) will be developed for N-Glycan detection kits, as well as optimal conditions for preparing and storage. Kits may feature affinity resins and columns for applications such as separating N-glycoproteins, enriching N-glycopeptides from tryptic digests of glycoproteins, and enriching N-glycoproteins from cell extracts.

The invention also includes conjugates of a binding reagent that specifically binds to N-glycosylated peptides and proteins as described herein. A conjugate includes, as a first component, the binding reagent, a Fbs variant polypeptide as described herein, which is covalently linked to at least one second component, which may be a proteinaceous component or a nonproteinaceous component. In some embodiments, a conjugate that includes a proteinaceous component can be synthesized as a fusion protein using well-known recombinant DNA methods. In some embodiments, the conjugate includes a proteinaceous or non-proteinaceous component that is chemically or enzymatically conjugated to the binding reagent.

One example of a conjugate of the invention includes a binding reagent conjugated to a therapeutic agent, also referred to herein as a drug. This conjugate is analogous to the well-known antibody-drug conjugate (ADC) except that the binding reagent is used in place of the antibody. Drugs that can be conjugated to a binding reagent include, without limitation, cytotoxins, anti-metabolites, alkylating agents, antibiotics, and anti-mitotic agents.

Anti-cancer, anti-inflammatory, pro-inflammatory, and immune-moderating drugs are particularly suitable for conjugation to a binding reagent, since cancerous and precancerous conditions, as well as inflammation and immune conditions, are often associated with changes in protein glycosylation patterns. For example, a therapeutic or diagnostic radioactive agent can be coupled to or incorporated into a binding reagent to target a cancer glycomarker. Likewise, anti-viral and anti-bacterial drugs are also particularly suitable for incorporation into conjugate, as targeting viral or bacterial glycosylated biomolecules has great therapeutic potential.

Another example of a binding reagent as described herein conjugated to a diagnostic or detection agent. The diagnostic or detection agent can include a detectable label, including but not limited to a radioactive, fluorescent, phosphorescent, colorimetric, enzymatic, immunological, magnetic, paramagnetic, diamagnetic, or electromagnetic label. It should be understood that a binding reagent need not be conjugated to function as a diagnostic or detection agent, as the binding reagent can be detected directly, e.g., via immunoassay.

Another example of a conjugate includes a binding reagent conjugated to a marker sequence. Such a marker includes, for example, an immobilization tag, including, but not limited to, a peptide such as hexa-histidine or hemagglutinin, to facilitate purification. Included in the invention are, for example, fusion proteins that include a binding reagent covalently linked to glutathione S-transferase (GST), thioredoxin, bovine serum albumin, bovine pancreatic trypsin inhibitor, or fluorescent proteins such as green fluorescent protein (GFP).

In another aspect, the disclosure provides an affinity matrix that includes an Fbs variant attached thereto. In accordance with this aspect, the affinity matrix is capable of specifically binding N-glycosylated proteins that can be eluted from the matrix and does not show substantial binding of non-glycosylated proteins. In a particular embodiment, the affinity matrix has an Fbs1 variant protein attached thereto, including but not limited to variants Fbs1-1, Fbs1-2, Fbs1-3, Fbs1-4, Fbs1-5, Fbs1-6, Fbs1-7, or Fbs1-8. Examples of an affinity matrix include, but are not limited to, a solid support, surface, column, resin, bead, particle, and nanoparticle. In another aspect, an affinity matrix as provided here includes an Fbs1 variant conjugate or Fbs1 variant fusion protein.

The present disclosure is directed to compositions and methods capable of specifically recognizing and binding to N-glycosylated peptides and proteins, and the provision of N-glycosylation-specific analytical, diagnostic, and therapeutic tools and reagents. A vast number of potential applications of a binding reagent that specifically binds to N-glycosylated peptides and proteins as described herein, because of its lectin-like properties, will be immediately apparent to persons skilled in the art. In general, a binding reagent or conjugate thereof, can be used for any of the same purposes for which anti-glycan antibodies are currently used. Thus, the compounds of the invention can be advantageously substituted for anti-glycan antibodies in numerous medical and laboratory methods, including diagnostic, analytical and therapeutic methods. Likewise, a binding reagent or conjugate thereof, can be used for the same purposes for which lectins are currently used. Thus, the compounds of the invention can be advantageously substituted for lectins in numerous diagnostic and analytical laboratory methods.

In some embodiments, a binding reagent or conjugate thereof, provides methods for enriching, isolating, and/or purifying an N-linked glycan or free N-glycan. The method can include contacting a biological or laboratory sample with an Fbs variant as provided herein, in particular Fbs1-1, Fbs1-2, Fbs1-3, Fbs1-4, Fbs1-5, Fbs1-6, Fbs1-7, or Fbs1-8, or a mixture thereof, under conditions that allow binding of the Fbs variant to an N-glycan, followed by steps that yield an enriched, isolated, and/or purified N-linked glycan or free N-glycan. In accordance with this aspect, the Fbs variant-bound N-glycan can be separated from the remainder of the sample and recovered in bound form or released from the Fbs variant to be recovered in free form. The N-glycan bound by the Fbs variant may optionally be isolated or purified. Alternatively, or in addition, the remainder of the sample may be recovered after binding, to obtain an N-glycan-depleted sample.

In accordance with another aspect, the present provides Fbs variants for use in compositions suitable for use as N-glycan-specific analytical, diagnostic, and therapeutic agents. Non-limiting embodiments include the use of an Fbs1 variant as described herein with a label, tag, or partner molecular for use as an analytical, diagnostic, and therapeutic agent. In certain embodiments, an Fbs1 variant can further comprise a detectable label such as a radioactive label, a fluorescent label, a phosphorescent label, a colorimetric label, an enzymatic label, an immunological label, a magnetic label, a paramagnetic label, a diamagnetic label, and an electromagnetic label. In some embodiments, an Fbs1 variant as described herein can further comprise a ligand, a drug, and a fusion protein partner. Also provided is a use of an Fbs variant as provided herein for targeted drug delivery, in particular to N-glycosylated targets.

Fbs variants as provided herein can be used to develop a pan-specific N-glycosylation detection reagent, which can provide the first pan-specific reagent able to detect/enrich all N-glycans and N-glycoconjugates. Such a reagent could simplify workflow, enhance the capacity to easily capture N-glycans for downstream analysis, and improve the likelihood of discovering uncommon disease-related glycans.

A binding reagent as described herein or conjugate thereof can be used to detect N-linked glycans (N-glycosylated proteins) in a biological or synthetic sample using an Fbs variant as provided herein, in particular Fbs1-1, Fbs1-2, Fbs1-3, Fbs1-4, Fbs1-5, Fbs1-6, Fbs1-7, or Fbs1-8, or a mixture thereof. The method can include contacting a biological or laboratory sample with an Fbs variant under conditions to allow binding of the Fbs variant to an N-glycan; and detecting the N-linked glycan. The method may further include characterizing the detected N-linked glycan, for example by identifying a constituent saccharide of the glycan, determining saccharide composition of the glycan, determining linkage positions within the glycan, or determining stereochemistry of the glycan.

For example, a biological sample, such as a tissue or fluid, can be contacted with the binding reagent or conjugate thereof to detect and/or characterize the level or type of glycosylation and/or glycation in the biological sample. Characterization of the glycan can include identifying a constituent saccharide of the glycan, determining saccharide composition of the glycan, determining linkage positions within the glycan, or determining stereochemistry of the glycan. As another example, a binding reagent or conjugate thereof can be used for quality control in the synthesis of therapeutic biologics, for example in the synthesis of therapeutic antibodies, to monitor the level or type of glycosylation. See, for example, PCT patent publication WO2012/118928. A binding reagent or conjugate thereof can be utilized as an affinity reagent or as part of an affinity matrix; for example, it can be tethered to a solid support, such as a surface, column, resin, bead, particle, or nanoparticle, and used in methods to detect or enrich for N-linked compounds in or from biological or synthetic samples. Tethered binding reagents can also be used to isolate and/or purify synthetic glycosylated compounds.

Diagnostics can be performed on a biological sample obtained from a subject but can also be performed in vivo. In in vivo applications, a binding reagent or conjugate thereof is administered to a subject, and binding of the binding reagent within the subject is detected. Preferably, a conjugate is administered to the subject, wherein the conjugate includes a detectable label so as to facilitate biomedical imaging. Examples of a suitable conjugate include a binding reagent conjugated to a radiolabel, a paramagnetic label, or a diamagnetic label.

A binding reagent described herein, with enhanced binding affinity toward N-linked glycans can be used to interrogate biological samples in the search for abnormal glycosylation. Examples of biological samples include, but are not limited to, any biological fluid, tissue, or organ. Examples of the biological fluids include, but are not limited to blood, urine, serum, saliva, cerebra-spinal fluid, and semen. In other embodiments, a binding reagent can be used for a detection of the presence or amount of a target analyte in biological fluids and tissues. Examples of such targets are exogenously consumed species, such as plant polysaccharides, carbohydrate-based drugs, and pathogens, whose surfaces are often coated in complex distinct glycans. The binding reagent also has application in drug discovery and evaluation of biological activity of new glycan-based compounds.

A binding reagent as described herein can be used for diagnosing, and/or treating diseases manifested by abnormal glycosylation. It can be used to detect certain tumor antigens comprising glycoproteins, glycolipids, and/or a variety of carbohydrate epitopes. A number of these tumor antigens have been found to be up-regulated in the neoplastic disease state.

Examples of tumor antigens that can signal a development and progression of a neoplastic disorder, and that can be detected with a binding reagent as described herein include, but are not limited to, carcinoembryonic antigen (CEA), which is a glycoprotein associated with colorectal, gastric, pancreatic, lung, and breast carcinomas, and the developing fetus; carbohydrate antigen 19-9 (CA 19-9), or sialylated Lewis A antigen, which is present in a glycolipid found in patients with pancreatic cancer; and carbohydrate antigen 15-3 (CA15-3), associated with breast cancer.

A binding reagent as described herein can be used in monitoring specific N-linked glycan modifications of proteins in biological fluids, tissues, organs, or living cells.

In yet other embodiments, a binding reagent can be used for in vitro or in vivo staining cells or tissues.

A binding reagent can be employed to monitor N-linked glycosylation in a mixture, as might arise during the production of recombinant glycoproteins for use in the pharmaceutical or research industries.

In the foregoing embodiments, the binding reagent can be tagged with a stain or a dye and applied to a biological sample comprising cells or tissues or glycoproteins or glycopeptides or oligosaccharides or polysaccharides of interest.

Another aspect of the present invention provides methods of using a binding reagent as described herein for analytical applications. The binding reagent can be used as an N-linked glycan-specific analytical tool. Glycan-specific analytical tools have potential use as a method of detection in many areas, including environmental, fermentation, food and medical areas and could be used for in vivo or in vitro sensing in humans or animals. For example, the binding reagent can be used as an affinity reagent or as vehicle for tissue staining. As another example, the binding reagent can be used for enriching a biological sample for N-linked glycans. In yet other examples, a binding reagent as described herein can be used to determine specific glycosylation sites on glycoproteins.

In certain embodiments, a binding reagent can be used as a reagent for affinity separation, including, for example, affinity chromatography. Affinity chromatography is a method of separating biochemical mixtures, based on a highly specific biological interaction such as that between the binding protein and the glycan. The present invention is not limited to any specific design or chromatographic system. In general, the binding reagent may be either covalently attached or otherwise immobilized to the solid support and will constitute a stationary phase. In certain embodiments, the stationary phase that is derivatized with the binding reagent can be used in column chromatography. In these embodiments, the particles of the solid stationary phase will be used to fill the whole inside volume of the tube (packed column). Alternatively, the solid phase particles will be concentrated on or along the inside tube wall leaving an open, unrestricted path for a biological sample (i.e., the mobile phase) in the middle part of the tube (open tubular column). In other embodiments, the derivatized stationary phase can be used for batch chromatography. In these embodiments, the stationary phase can be added to a vessel and mixed with the biological sample. Although the foregoing example generally focused on affinity chromatography, it is understood that these principals are readily applied to other affinity purification protocols.

In certain embodiments, a binding reagent can be used as a therapeutic agent or modified for delivery of an active therapeutic agent. Since the binding reagent has a specificity for N-linked glycans, the delivery of the therapeutic agent can be targeted to cells, tissues, or organs that N-linked glycans. An N-linked glycans or conjugate thereof, can be administered to a subject to treat or prevent an infection, disease, or disorder. The infection can be, for example, viral, bacterial, parasitic, or fungal. The disease or disorder can result from an exogenous agent, or it can be autologous or autoimmune.

In another aspect, the disclosure provides an isolated polynucleotide encoding an Fbs variant, a proteinaceous conjugate thereof, or a fusion protein as provided herein. Also provided is a vector that includes or incorporates the polynucleotide. The vector can be an expression vector or a cloning vector. The invention further provides a host cell that includes said vector. The host cell can be a bacterial cell, a fungal cell (such as yeast) or an animal cell, such as an insect or a mammalian cell. Examples of a suitable host cell include an Escherichia coli cell or a yeast cell, such Saccharomyces cerevisiae.

In another aspect, the disclosure provides a method for making an Fbs variant as provided herein, in particular a Fbs1-1, Fbs1-2, Fbs1-3, Fbs1-4, Fbs1-5, Fbs1-6, Fbs1-7, or Fbs1-8 variant. An Fbs variant can be expressed from an isolated polynucleotide, expression vector, or host cell, in vitro or in vivo.

A binding reagent as described herein may be expressed in a host cell using genetic engineering techniques. The term “cell” is meant to include any type of biological cell. The host cell can be a eukaryotic cell or a prokaryotic cell. Preferably, the host cell is a prokaryotic cell such as a bacterial cell; however single cell eukaryotes such as protists or yeasts are also useful as host cells. Preferred host cells are microbial cells, preferably the cells of single-celled microbes such as bacterial cells or yeast cells. Notwithstanding the above preferences for bacterial and/or microbial cells, it should be understood that the binding reagent can be expressed without limitation in the cell of an animal, plant, insect, yeast, protozoan, bacterium, or archaebacterium. Examples of microbial cells that can be engineered to express the binding reagent, in addition to E. coli, include a wide variety of bacteria and yeast including members of the genera Escherichia, Salmonella, Clostridium, Zymomonas, Pseudomonas, Bacillus, Rhodococcus, Alcaligenes, Klebsiella, Paenibacillus, Lactobacillus, Enterococcus, Arthrobacter, Brevibacterium, Corynebacterium Candida, Hansenula, Pichia and Saccharomyces. Preferred microbial cells include, without limitation, Escherichia coli, Bacillus subtilis, Bacillus licheniformis, Alcaligenes eutrophus, Rhodococcus erythropolis, Paenibacillus macerans, Pseudomonas putida, Enterococcus faecium, Saccharomyces cerevisiae, Lactobacillus plantarum, Enterococcus gallinarium, and Enterococcus faecalis.

A cell that has been genetically engineered to express a binding reagent as described herein may be referred to as a “host” cell, a “recombinant” cell, a “genetically engineered” cell or simply an “engineered” cell. These and similar terms are used interchangeably. A genetically engineered cell contains one or more artificial sequences of nucleotides which have been created through standard molecular cloning techniques to bring together genetic material that is not natively found together. DNA sequences used in the construction of recombinant DNA molecules can originate from any species. For example, plant DNA may be joined to bacterial DNA, or human DNA may be joined with fungal DNA. Alternatively, DNA sequences that do not occur anywhere in nature may be created by chemical synthesis of DNA or by directed mutation of DNA and incorporated into recombinant molecules. Proteins that result from the expression of recombinant DNA are often termed recombinant proteins. Examples of recombination are described in more detail below and may include inserting foreign polynucleotides (obtained from another species of cell) into a cell, inserting synthetic polynucleotides into a cell, or relocating or rearranging polynucleotides within a cell. Any form of recombination may be considered to be genetic engineering and therefore any recombinant cell may also be considered to be a genetically engineered cell.

As will be appreciated by a person of skill in the art, expression of a protein, such as a binding reagent as described herein, can be achieved through a number of molecular biology techniques. For example, expression can be achieved by introducing into the host cell one or more copies of a polynucleotide encoding the desired protein. The polynucleotide encoding the desired protein may be endogenous or heterologous to the host cell. Preferably, the polynucleotide is introduced into the cell using a vector; however, naked DNA may also be used. The polynucleotide may be circular or linear, single-stranded, or double stranded, and can be DNA, RNA, or any modification or combination thereof. The vector can be any molecule that may be used as a vehicle to transfer genetic material into a cell. Examples of vectors include plasmids, viral vectors, cosmids, and artificial chromosomes, without limitation. Examples of molecular biology techniques used to transfer nucleotide sequences into a microorganism include, without limitation, transfection, electroporation, transduction, and transformation. These methods are well known in the art. Insertion of a vector into a target cell is usually called transformation for bacterial cells and transfection for eukaryotic cells, however insertion of a viral vector is often called transduction. The terms transformation, transfection, and transduction, for the purpose of the instant invention, are used interchangeably herein. A polynucleotide which has been transferred into a cell via the use of a vector is often referred to as a transgene.

Preferably, the vector is an expression vector. An “expression vector” or “expression construct” is any vector that is used to introduce a specific polynucleotide into a target cell such that once the expression vector is inside the cell, the protein that is encoded by the polynucleotide is produced by the cellular transcription and translation machinery. Typically, an expression vector includes regulatory sequences operably linked to the polynucleotide encoding the desired protein. Regulatory sequences are common knowledge to the person of the skill in the art and may include for example, an origin of replication, a promoter sequence, and/or an enhancer sequence. The polynucleotide encoding the desired protein can exist extrachromosomally or can be integrated into the host cell chromosomal DNA.

Extrachromosomal DNA may be contained in cytoplasmic organelles, such as mitochondria (in most eukaryotes), and in chloroplasts and plastids (in plants). More typically, extrachromosomal DNA is maintained within the vector on which it was introduced into the host cell. In many instances, it may be beneficial to select a high copy number vector in order to maximize the expression of the protein. Optionally, the vector may further contain a selectable marker. Certain selectable markers may be used to confirm that the vector is present within the target cell. Other selectable markers may be used to further confirm that the vector and/or transgene has integrated into the host cell chromosomal DNA. The use of selectable markers is common in the art and the skilled person would understand and appreciate the many uses of selectable markers. Optionally, the vector may further contain a reporter gene. Reporter genes may be used to confirm that the vector is expressing within the target cell and may be further used to monitor the expression from the vector. The use of reporter genes is common in the art and the skilled person would understand and appreciate the many uses of reporter genes.

A binding reagent as described herein can be isolated and optionally purified from any genetically engineered cell described herein. It can be isolated directly from the cells, or from the culture medium, for example, during an aerobic or anaerobic fermentation process. Isolation and/or purification can be accomplished using known methods.

Also provided by the invention is a kit that includes one or more of the binding reagents described herein, a conjugate thereof, a fusion protein thereof, or an affinity matrix thereof, and instructions for use. Non-limiting embodiments include a kit that includes a Fbs1-1, Fbs1-2, Fbs1-3, Fbs1-4, Fbs1-5, Fbs1-6, Fbs1-7, and/or Fbs1-8 variant as described herein. Optionally the kit can include buffers, salts, labeling or detection reagents, or other diagnostic or analytical reagents.

### Exemplary Aspects

The invention is defined in the claims. However, below there is provided a non-exhaustive listing of non-limiting exemplary aspects. Any one or more of the features of these aspects may be combined with any one or more features of another example, embodiment, or aspect described herein. Exemplary Embodiments of the present invention include, but are not limited to, the following.

Aspect 1 includes a binding reagent that specifically binds to N-glycosylated peptides and proteins and does not show substantial binding to non-glycosylated peptides and proteins, wherein the binding reagent comprises an amino acid sequence having at least 90% identity to amino acid residues 125 to 297 (the sugar binding domain) of the murine Fbs1 protein (SEQ ID NO: 9) and having an amino acid substitution at position 153 and/or 154 of SEQ ID NO:9.

Aspect 2 is the binding reagent of Aspect 1 further comprising an amino acid sequence having at least 90% sequence identity to amino acid residues 96 to 124 (the linker domain) of the murine Fbs1 protein (SEQ ID NO: 9).

Aspect 3 is the binding reagent of Aspect 1 or 2 further comprising a N-terminal immobilization tag.

Aspect 4 is the binding reagent of Aspect 3 wherein the N-terminal immobilization tag comprises the amino acid sequence MGSSHHHHHHHHSSGENLYFQSHMAS (SEQ ID NO: 19).

Aspect 5 is the binding reagent of any one of Aspects 1 to 4, wherein the substitution at position 153 and/or 154 comprises a E153R and/or a E154R substitution.

Aspect 6 is the binding reagent of any one of Aspects 1 to 5 further comprising an amino acid substitution at position 177 and/or 178.

Aspect 7 is the binding reagent of Aspect 6 wherein the substitution at position 177 and/or 178 comprises a F177Y and/or a E178R substitution.

Aspect 8 is the binding reagent protein of any one of Aspects 1 to 7 further an comprising amino acid substitution at position 158 and/or 159.

Aspect 9 is the binding reagent of Aspect 6 further comprising an amino acid substitution at position 158 and/or 159.

Aspect 10 is the binding reagent of Aspect 8 or 9 wherein the substitution at position 159 comprises a N159G substitution.

Aspect 11 is the binding reagent of any one of Aspects 1 to 10, wherein the binding reagent comprises at least 90% sequence identity to SEQ ID NO: 6, SEQ ID NO: 7, SEQ ID NO: 8, SEQ ID NO: 16, SEQ ID NO: 17, or SEQ ID NO: 18.

Aspect 12 is a composition comprising the binding reagent of any one of Aspects 1 to 11.

Aspect 13 is a conjugate comprising a first component comprising a binding reagent of any one of Aspects 1 to 12 covalently linked to a second component.

Aspect 14 is an affinity matrix comprising the binding reagent of any one of Aspects 1 to 13.

Aspect 15 is the affinity matrix of Aspect 14 selected from the group consisting of a solid support, surface, a column, a resin, a bead, a particle, and a nanoparticle.

Aspect 16 is an isolated polynucleotide encoding the binding reagent of any one of Aspects 1 to 11.

Aspect 17 is a vector comprising the polynucleotide of Aspect 16.

Aspect 18 is a host cell comprising the polynucleotide of Aspect or the vector of Aspect claim 17.

Aspect 19 is a method for detecting an N-glycosylated protein in a sample, the method comprising contacting the sample with a binding reagent, composition, conjugate, or affinity matrix of any one of Aspects 1 to 15 under conditions to allow binding of the binding reagent to the N-glycosylated protein and detecting the N-glycosylated protein bound to the binding reagent.

Aspect 20 is a method for enriching, isolating, or purifying an N-glycosylated protein, the method comprising contacting a biological or laboratory sample with a binding reagent, composition, conjugate, or affinity matrix of any one of Aspects 1 to 15 under conditions to allow binding of the binding reagent to an N-glycosylated protein in the sample, and recovering the N-glycosylated protein bound to the binding reagent.

Aspect 21 is the method of Aspect 20, further comprising separating the recovered N-glycosylated protein from the binding reagent.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Mouse Fbs1 Variants

When wild-type mouse (Mus musculus) mmFBXO2 sequence (SEQ ID NO: 9) was compared to human (Homo sapiens) F-Box only protein 2 (hsFBXO2 (SEQ ID NO: 20), Chen et al., 2017, Nature Comm, 8;15487) using EMBOSS Needle (a pairwise sequence alignment software that can be accessed on the worldwide web at ebi.ac.uk/Tools/psa/emboss_needle/), the alignment showed that the mmFBXO2 sequence (SEQ ID NO: 9) shares only an 87% identity in amino sequence with its human counterpart hsFBXO2 (SEQ ID NO: 20). This is shown in FIG. 1A.

### Fbs1 Variants 1-1 and 1-2

Two Fbs1 variants were designed, expressed in Escherichia coli, purified by immobilized metal affinity chromatography with a yield of about 0.25 mg/L, and designated Fbs1-1 and Fbs1-2. Fbs1 variants Fbs1-1 and Fbs1-2 were tested and N-glycan-specific binding was demonstrated for both variants (see Examples 2-8 below). Each variant was able to specifically bind to N-glycosylated peptides and proteins and did not show substantial binding to non-glycosylated peptides and proteins. Differences between the Fbs1 variants were observed for parameters such as signal strength, or affinity for different ligands, which indicated that the variants had potential for tunable affinities and binding kinetics, and tunable specificities that could be focused to develop desired properties of specific utility in one or more application such as affinity chromatography, flow cytometry, immunohistochemistry, Western blot analysis, purification, sample enrichment, or for therapeutic and/or diagnostic applications.

The two Fbs1 variants have the following amino acid sequences.

Variant Fbs1-1
(SEQ ID NO: 1)
MGSSHHHHHHHHSSGENLYFQSHMASQQEGLVPEGSADEERDHWQQFYFL
SKRRRNLLRNPCGEEDLEGWSDVEHGGDGWRVEELPGDNGVEFTQDDSVK
KYFASSFEWCRKAQVIDLQAEGYWEELLDTTQPAIVVKDWYSGRTDAGSL
YELTVRLLSENEDVLAEFATGQVAVPEDGSWMEISHTFIDYGPGVRFVRF
EHGGQDSVYWKGWFGARVTNSSVWVEP

The Fbs1-1 variant without the N-terminal immobilization tag has the following sequence:

(SEQ ID NO: 11)
QQEGLVPEGSADEERDHWQQFYFLSKRRRNLLRNPCGEEDLEGWSDVEHG
GDGWRVEELPGDNGVEFTQDDSVKKYFASSFEWCRKAQVIDLQAEGYWEE
LLDTTQPAIVVKDWYSGRTDAGSLYELTVRLLSENEDVLAEFATGQVAVP
EDGSWMEISHTFIDYGPGVRFVRFEHGGQDSVYWKGWFGARVTNSSVWVE
P

Variant Fbs1-2
(SEQ ID NO: 2)
MGSSHHHHHHHHSSGENLYFQSHMASQQEGLVPEGSADEERDHWQQFY
FLSKRRRNLLRNPCGEEDLEGWSDVEHGGDGWRVEELPGDNGVEFTQD
DSVKKYFASSYRWCRKAQVIDLQAEGYWEELLDTTQPAIVVKDWYSGR
TDAGSLYELTVRLLSENEDVLAEFATGQVAVPEDGSWMEISHTFIDYG
PGVRFVRFEHGGQDSVYWKGWFGARVTNSSVWVEP

The Fbs1-2 variant without the N-terminal immobilization tag has the following sequence:

(SEQ ID NO: 12)
QQEGLVPEGSADEERDHWQQFYFLSKRRRNLLRNPCGEEDLEGWSDVE
HGGDGWRVEELPGDNGVEFTQDDSVKKYFASSYRWCRKAQVIDLQAEG
YWEELLDTTQPAIVVKDWYSGRTDAGSLYELTVRLLSENEDVLAEFAT
GQVAVPEDGSWMEISHTFIDYGPGVRFVRFEHGGQDSVYWKGWFGARV
TNSSVWVEP

### Example 2

### Binding Specificity of Fbs Variants Fbs1-1 and Fbs1-2 Using Biolayer Interferometry (BLI)

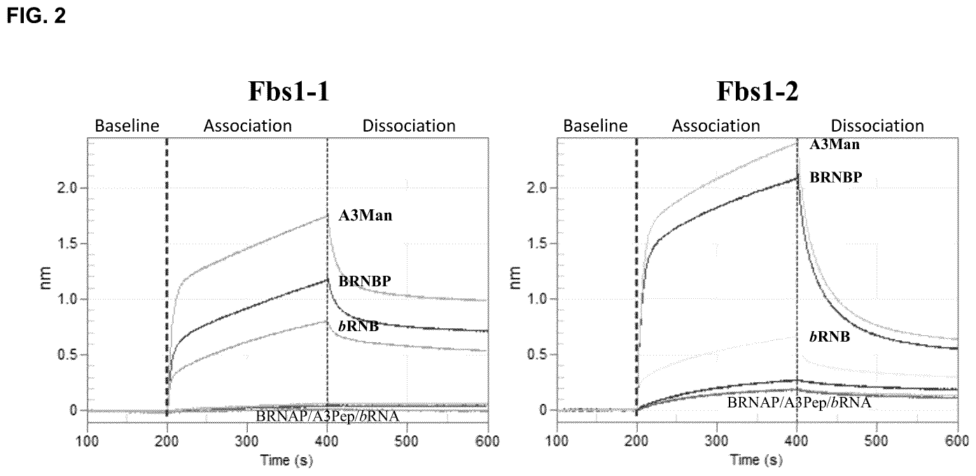

Biolayer Interferometry (BLI) was used to measure the specificity of Fbs variants Fbs1-1 and Fbs1-2 binding to biotinylated ligands immobilized on SAX biosensors. FIG. 2 shows results for Fbs1-1 (left panel) and Fbs1-2 (right panel) binding to: biotinylated N-glycopeptide A3Man (AVSHN(M3N2)ASLE); biotinylated N-glycopeptide BRNBP (SRN(M3N2)LTK); biotinylated N-glycosylated RNase B (bRNB); biotinylated non-glycosylated peptide A3Pep (AVSHNASLE); biotinylated non-glycosylated peptide BRNAP (SRNLTK); and biotinylated non-glycosylated peptide RNase A (bRNA). Transient binding constants are based on the 2:1 curve fitting model.

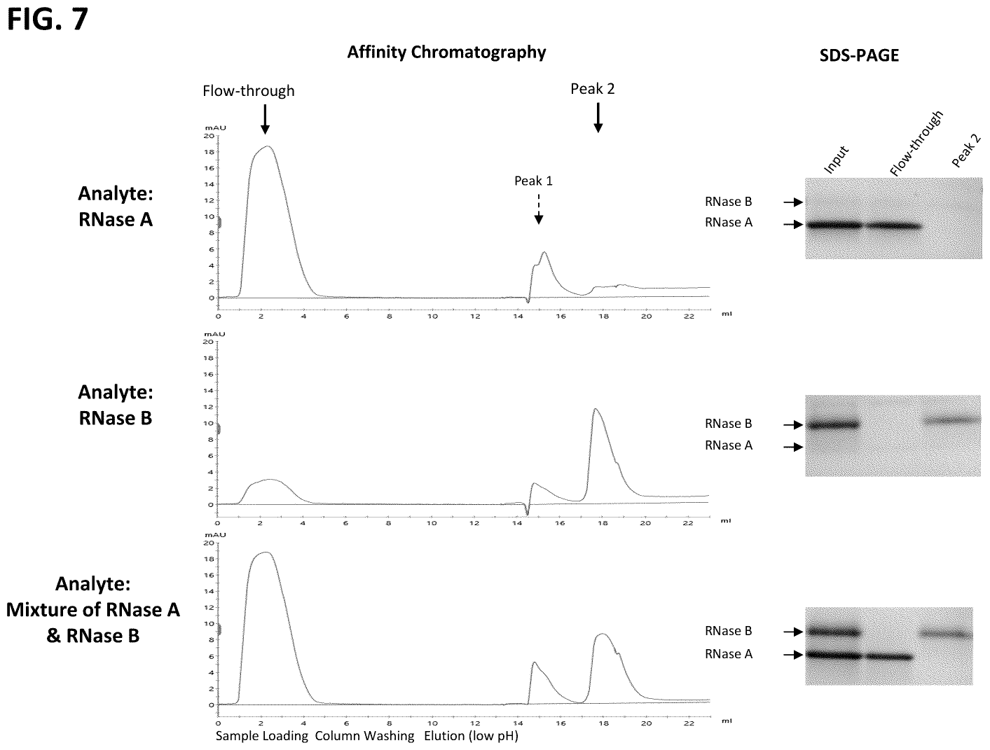

The results shown in FIG. 2 demonstrate that both Fbs1 variants, Fbs1-1 and Fbs1-2, bind to N-glycopeptides A3Man and BRNBP, and to N-glycosylated RNase B, but not to non-glycosylated peptides A3Pep and BRNAP, and not to non-glycosylated RNase A. The results shown in FIG. 2 further demonstrate that Fbs1-2 shows a higher level of response to the immobilized ligands than Fbs1-1. These results indicate that mutagenesis of Fbs1 can result in binding affinity that is tunable for the desired application, e.g., such as for affinity chromatography as in Example 8 and FIG. 7.

### Example 3

### Western Blot Analysis of Glycoproteins Using Variants Fbs1-1 and Fbs1-2

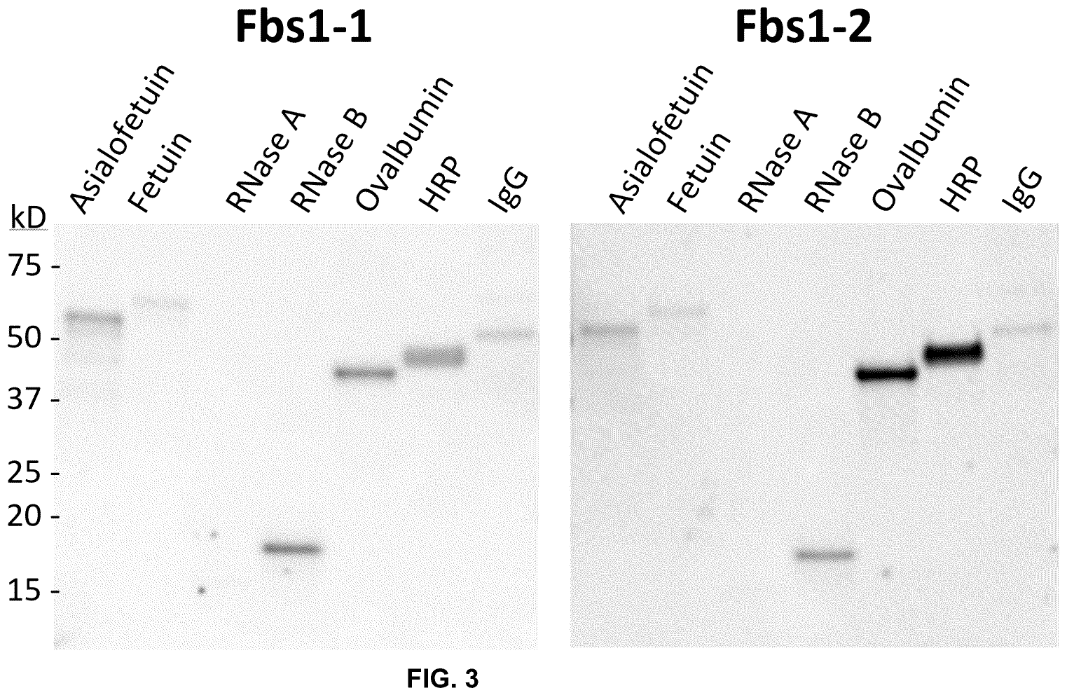

Samples (1 μg each) of glycoproteins (Asialofetuin, Fetuin, RNase B, Ovalbumin, Horse Radish Peroxidase, and IgG (polyclonal antibody from rabbit)) and a non-glycosylated protein (RNase A) were separated by SDS-PAGE, blotted on nitrocellulose membrane, and probed by Fbs1-1 or Fbs1-2. The results shown in FIG. 3 demonstrate that both Fbs1 variants are capable of binding to all of the glycoproteins tested and do not bind to non-glycosylated RNase A (the non-glycosylated form of RNase B. The left panel of FIG. 3 shows that Fbs1-1 bound to Asialofetuin, Fetuin, RNase B, Ovalbumin, Horse Radish Peroxidase, and IgG and did not bind to non-glycosylated RNase A (the non-glycosylated form of RNase B). The right panel of FIG. 3 shows that Fbs1-2 bound to Asialofetuin, Fetuin, RNase B, Ovalbumin, Horse Radish Peroxidase, and IgG and did not bind to non-glycosylated RNase A (the non-glycosylated form of RNase B). Comparison of the results for glycoprotein RNase B and non-glycosylated RNase A in each blot shows that neither Fbs1 variant showed non-specific binding to the RNase molecule. As shown in FIG. 3, the Fbs1-2 signal (right panel) is stronger on ovalbumin and HRP than the Fbs1-1 signal for the same amount of ovalbumin and HRP, indicating Fbs1-2 may be a more sensitive probe than Fbs1-1. Furthermore, binding to HRP is significant as the N-glycans on HRP are core α1,3-fucosylated. Therefore, the Fbs1 variants detects both core α1,3-fucosylated and core α1,6-fucosylated N-glycans (see Example 5).

### Example 4

### Binding Specificity of Fbs1-1 Using ELISA

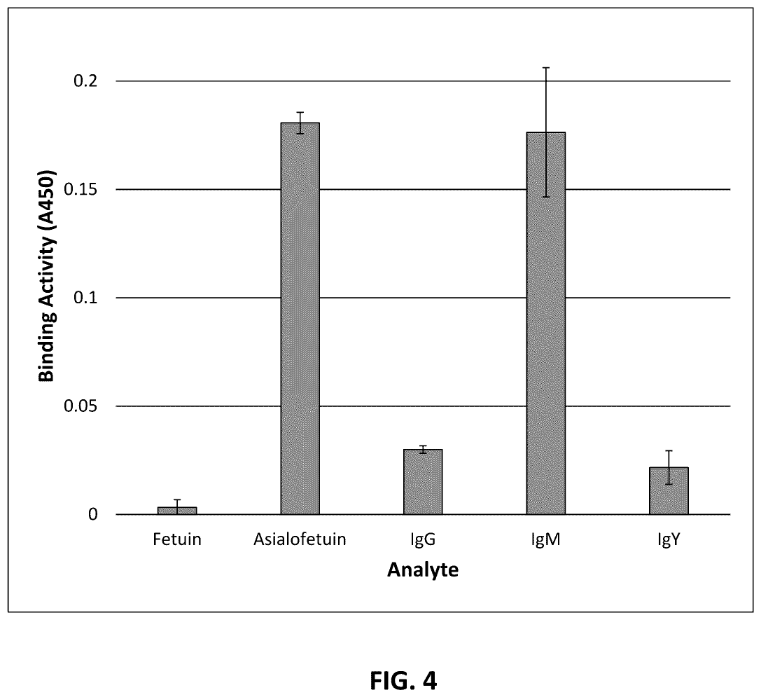

Plates were coated with 10 μg/mL of glycoprotein analytes fetuin. asialofetuin, rabbit IgG, mouse IgM, or chicken IgY, each plate was probed with 20 μg/mL Fbs1-1 in TBS buffer, and results were measured with TMB substrate through an anti-poly-His HRP conjugate. Fetuin is a heavily sialylated glycoprotein isolated from bovine serum; Asialofetuin is the desialylated form of Fetuin, IgG is a polyclonal immunoglobulin raised in rabbit, IgM an immunoglobulin raised in mouse, and IgY an immunoglobulin raised in chicken. The results shown in FIG. 4 demonstrate that Fbs1-1 showed preferential binding to desialylated Asialofetuin and IgM. (Moh et al., 2016, J Am Soc Mass Spectrom, 27(7):1143-55; Cobb, 2020, Glycobiology, 30(4):202-213; and Zlatina and Galuska, 2021, Front Immunol, 12:753294).

### Example 5

### Glycan Array Screening of Fbs1-1

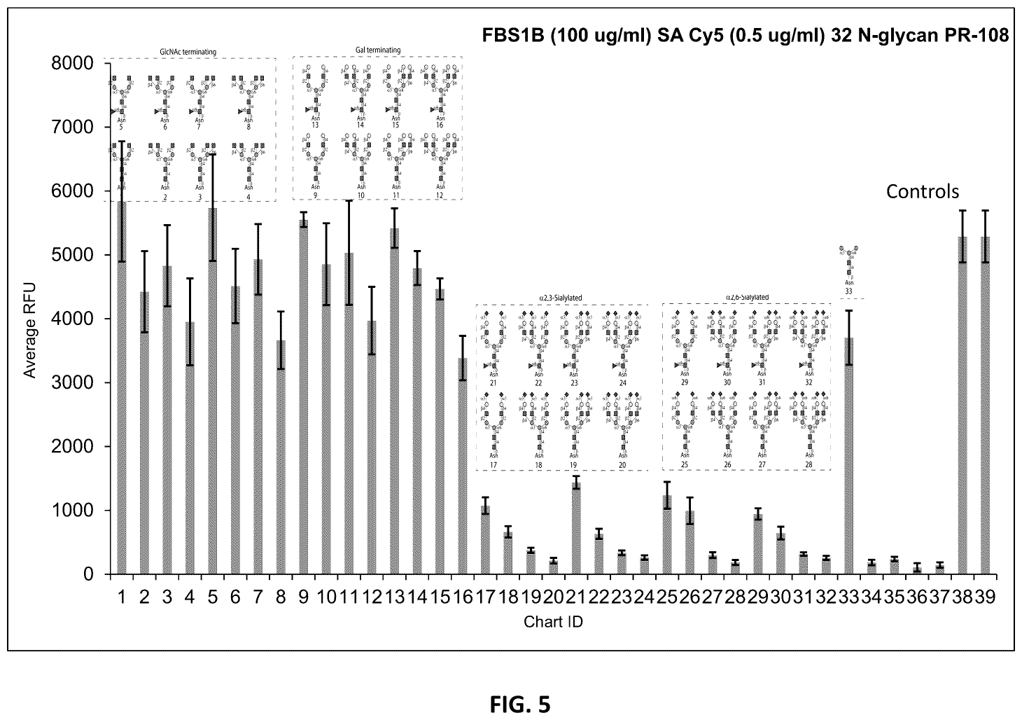

An array of immobilized glycans with structures as shown in FIG. 5 (structures from Gao et al., 2019, Cell Chem Biol; 26(4): 535-547) was incubated with 100 μg/mL biotinylated Fbs1-1, followed by 0.5 μg/mL of a streptavidin Cy5-635 conjugate. As shown in FIG. 5, Fbs1-1 showed a preference for terminal GlcNAc, Gal, and Man N-glycans over terminally sialylated glycans. These results show that variant Fbs1-1 (a truncated version of native mouse Fbs1 ) preferentially binds bi-, tri-, tetra-antennary non-sialylated glycans, with or without core α1,6-fucose.

### Example 6

### Binding Specificity of Fbs1-1 Using Yeast Display Assay

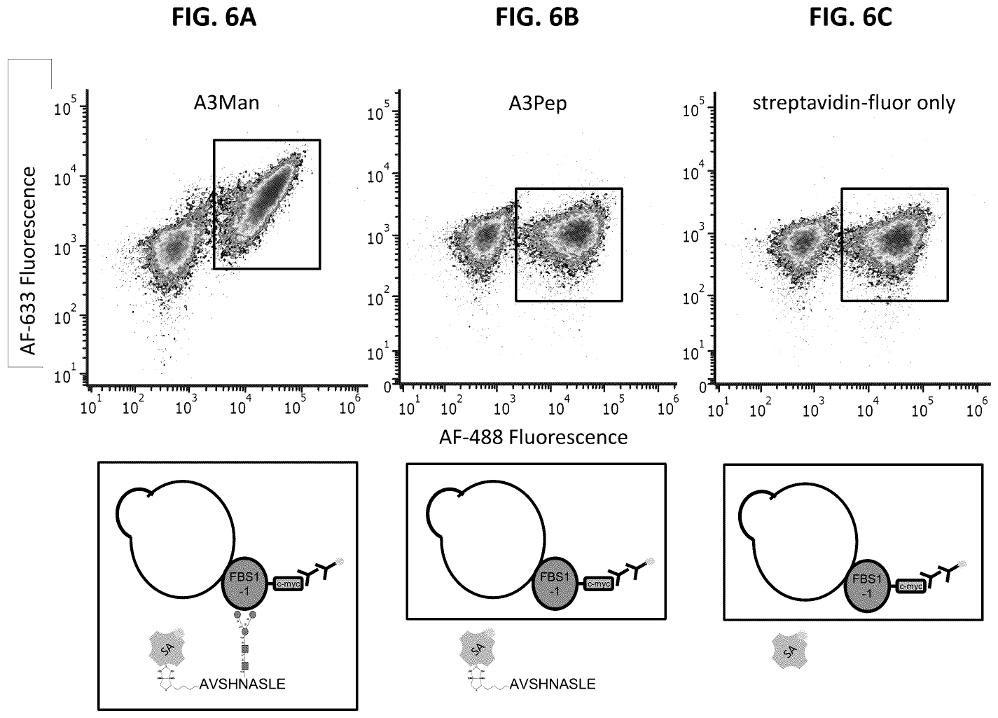

Yeast cells displaying Fbs1-1 on the cell surface were dual labeled with anti-c-Myc tag mouse mAb, followed by goat anti-mouse-Alexa Fluor 488 and either (a) the A3Man N-glycopeptide ligand precomplexed with streptavidin-Alex Fluor 633, (b) the A3Pep non-glycosylated peptide precomplexed with streptavidin-Alexa Fluor 633, or (c) the streptavidin-Alexa Fluor 633-only control. After incubation, cells were washed, sorted by fluorescence-activated cell sorting (FACS), and imaged. FACS results are shown in the upper row, and a simplified schematic of the interaction is shown in the bottom row. The results as shown in FIGS. 6A-6C demonstrated that only N-glycopeptide (A3Man N-glycopeptide) binds to Fbs-1-1 displayed on yeast surface as shown in panel (a), but not the corresponding non-glycosylated peptide (A3Pep non-glycosylated peptide) does not bind to Fbs1-1 displayed on yeast surface as shown in panel (b). These results indicated that Fbs1-1 is amenable to directed evolution using yeast display technology.

### Example 7

### Binding Specificity of Fbs1-1: Bio-panning Using Yeast Display Biocombinatorial Libraries

Yeast display biocombinatorial libraries are used for bio-panning on immobilized target by magnetic-activated cell sorting, and for as well as by fluorescence-activated cell sorting. Initial rounds of bio-panning of the library are performed against magnetic resin coated with an N-glycan target derived from HRP. Negative selection is performed on naked magnetic resin or resin coupled with de-glycosylated target. Beginning the bio-panning on resin is used to ensure capture of the most diverse set of binders, including low affinity clones. After enrichment on target-coupled magnetic resin, bio-panning can be alternated with flow cytometric sorting, which allows for direct selection of the highest affinity clones. This is accomplished by allowing the yeast to bind to a fluorescently labeled glycan target, and counter-staining against the HA or c-myc epitope tags present on the displayed construct. Individual yeast cells, positive for both fluorophores, are sorted into plates, grown, and sequenced. Clones of interest are confirmed by flow cytometric staining with labeled target, and recombinantly expressed in E. coli.

### Example 8

### Fbs1-1 Affinity Column Separates Non-N-Glycosylated Protein From N-Glycosylated Protein

Fbs1-1 (5mg) was chemically coupled to a 1-mL Sepharose column to generate an Fbs1. Three different analytes were evaluated using the Fbs1-1 affinity column: a glycoprotein, RNase B; its non-glycosylated counterpart, RNase A; and a 1:1 mixture of RNase A and RNase B. Each analyte was loaded on the column in TBS buffer, and the protein content of column output was monitored during column loading, column washing, and elution (low pH). Bound protein was eluted non-competitively with a low pH buffer (0.1M Glycine-HCl and 0.5M NaCl, pH 4.0). FIG. 7 shows the elution profile for each analyte, with labels showing fractions (peaks) of interest. Fractions of interest (including the flow-through (unbound materials) fraction, and apparent peak fractions) were collected and analyzed by SDS-PAGE and staining to confirm recovery of analytes and to confirm separation of RNase B from RNase A. As shown in FIG. 7 (right side, “SDS-PAGE”) a sample of the original analyte (lane labelled “Input”), the collected flow-through (lane labelled “Flow-through”) and the fraction corresponding to the elution peak (lane labelled “Peak 2”) and were analyzed by SDS-PAGE to confirm what was applied, what was bound, what did not bind, and separation of RNase B from RNase A.

The results shown in FIG. 7 confirm that Fbs1-1 selectively bound glycoprotein and did not show substantial binding of non-glycosylated protein and can be used to separate glycoproteins from non-glycosylated proteins. Results using RNase B as a glycoprotein analyte (lane labelled “Input” in middle panel of FIG. 7) demonstrate that the Fbs1-1 affinity matrix bound the glycoprotein RNase B, as the glycoprotein was collected in the elution fraction (lane labelled “Peak 2”) and was not detected in the unbound fraction (lane labelled “Flow-through”). Results using RNase A as a non-glycosylated-protein analyte (lane labelled “Input” in top panel of FIG. 7) demonstrate that the Fbs1-1 affinity matrix did not bind the non-glycosylated RNase A, which passed through the column and was collected in the flow-through fraction (lane labelled “Flow-through”) and did not appear in the elution fraction (lane labelled “Peak 2”). Results using a mixture of RNase A and RNase B as a mixture of glycoproteins and corresponding non-glycosylated protein (lane labelled “Input” in bottom panel of FIG. 7) shows that the Fbs1-1 affinity matrix was able to selectively bind the glycoprotein RNase B (lane labelled “Peak 2”) while the non-glycosylated RNase A did not bind (lane labelled “Flow through”). Note that the peak indicated as “Peak 1” on the elution profile for each analyte is an artifact resulting from the change from loading buffer (TBS) to the glycine elution buffer. As it was previously determined not to contain any protein, the “Peak 1” fraction is not shown on any SDS-PAGE gel.

### Example 9

### Variant Fbs1-3

For the Fbs1-3 variant, an additional mutation was added to the Fbs1-2 variant. Thus, in addition to the change of amino acids at positions 177 and 178 from FE to YR, amino acid position 159 is mutated from N to G. The amino sequence of Fbs1-3 is shown below:

Variant Fbs1-3
(SEQ ID NO: 3)
MGSSHHHHHHHHSSGENLYFQSHMASQQEGLVPEGSADEERDHWQQFY
FLSKRRRNLLRNPCGEEDLEGWSDVEHGGDGWRVEELPGDGGVEFTQD
DSVKKYFASSYRWCRKAQVIDLQAEGYWEELLDTTQPAIVVKDWYSGR
TDAGSLYELTVRLLSENEDVLAEFATGQVAVPEDGSWMEISHTFIDYG
PGVRFVRFEHGGQDSVYWKGWFGARVTNSSVWVEP

The Fbs1-3 variant without the N-terminal immobilization tag has the following sequence:

(SEQ ID NO: 13)
QQEGLVPEGSADEERDHWQQFYFLSKRRRNLLRNPCGEEDLEGWSDVE
HGGDGWRVEELPGDGGVEFTQDDSVKKYFASSYRWCRKAQVIDLQAEG
YWEELLDTTQPAIVVKDWYSGRTDAGSLYELTVRLLSENEDVLAEFAT
GQVAVPEDGSWMEISHTFIDYGPGVRFVRFEHGGQDSVYWKGWFGARV
TNSSVWVEP

### Example 10

### Variants Fbs1-6 Thru Fbs-1-8

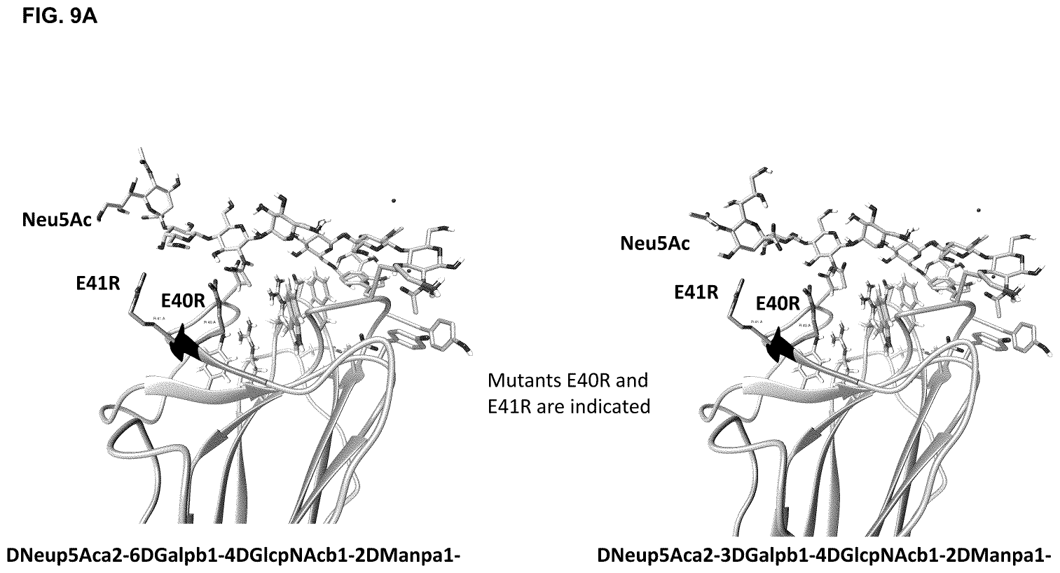

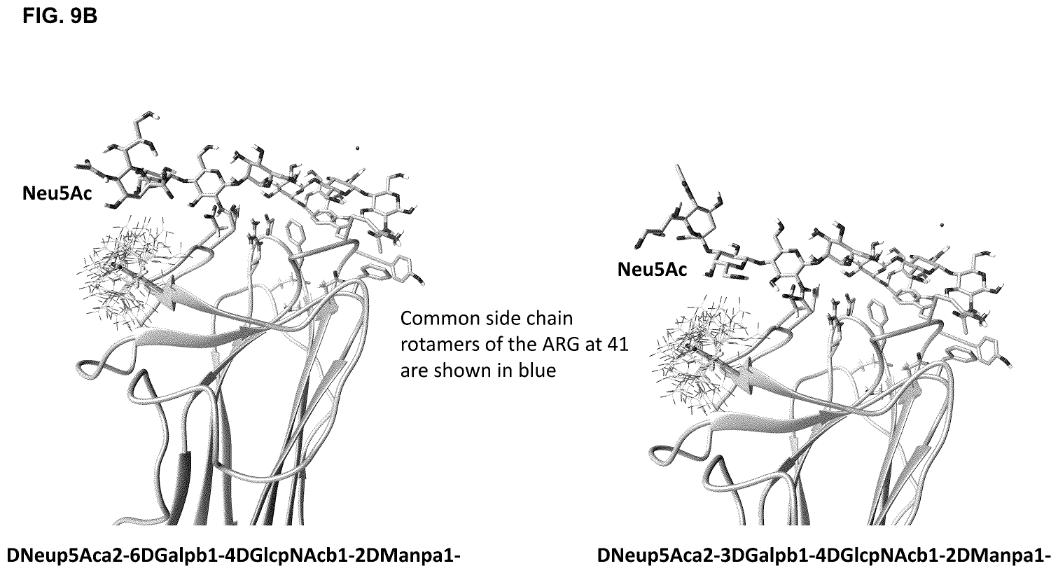

For the Fbs1-6, Fbs1-7, and Fbs1-8 variants, additional substitutions were introduced into the Fbs1-3 variant at positions 153 and/or 154. Thus, in addition to the FE to YR substitution at amino acids positions 177 and 178 and the N to G substitution at position 159, the Fbs1-6, Fbs1-7, and Fbs1-8 variants have additional substitutions at positions 153 and/or 154. Specifically, the Fbs1-6 variant has an E to R substitution at position 153, the Fbs1-7 variant has an E to R substitution at position 154, and the Fbs1-8 variant has the E to R substitution at both residues 153 and 154. Modelling shown in FIGS. 9A and 9B identified these residues for enhanced binding to sialylated N-glycans. Future variants may include the equivalent Lys mutants, ie KE, EK, KK, and possibly the equivalent polar neutral mutants (Q, N, H). The sequences of Fbs1-6, Fbs1-7, and Fbs1-8 variants are shown below:

Variant Fbs1-6
(SEQ ID NO: 6)
MGSSHHHHHHHHSSGENLYFQSHMASQQEGLVPEGSADEERDHWQQFY
FLSKRRRNLLRNPCGEEDLEGWSDVEHGGDGWRVRELPGDGGVEFTQD
DSVKKYFASSYRWCRKAQVIDLQAEGYWEELLDTTQPAIVVKDWYSGR
TDAGSLYELTVRLLSENEDVLAEFATGQVAVPEDGSWMEISHTFIDYG
PGVRFVRFEHGGQDSVYWKGWFGARVTNSSVWVEP.

The Fbs1-6 variant without the N-terminal immobilization tag has the following sequence:

(SEQ ID NO: 16)
QQEGLVPEGSADEERDHWQQFYFLSKRRRNLLRNPCGEEDLEGWSDVE
HGGDGWRVRELPGDGGVEFTQDDSVKKYFASSYRWCRKAQVIDLQAEG
YWEELLDTTQPAIVVKDWYSGRTDAGSLYELTVRLLSENEDVLAEFAT
GQVAVPEDGSWMEISHTFIDYGPGVRFVRFEHGGQDSVYWKGWFGARV
TNSSVWVEP.

Variant Fbs1-7
(SEQ ID NO: 7)
MGSSHHHHHHHHSSGENLYFQSHMASQQEGLVPEGSADEERDHWQQFY
FLSKRRRNLLRNPCGEEDLEGWSDVEHGGDGWRVERLPGDGGVEFTQD
DSVKKYFASSYRWCRKAQVIDLQAEGYWEELLDTTQPAIVVKDWYSGR
TDAGSLYELTVRLLSENEDVLAEFATGQVAVPEDGSWMEISHTFIDYG
PGVRFVRFEHGGQDSVYWKGWFGARVTNSSVWVEP.

The Fbs1-7 variant without the N-terminal immobilization tag has the following sequence:

(SEQ ID NO: 17)
QQEGLVPEGSADEERDHWQQFYFLSKRRRNLLRNPCGEEDLEGWSDVE
HGGDGWRVERLPGDGGVEFTQDDSVKKYFASSYRWCRKAQVIDLQAEG
YWEELLDTTQPAIVVKDWYSGRTDAGSLYELTVRLLSENEDVLAEFAT
GQVAVPEDGSWMEISHTFIDYGPGVRFVRFEHGGQDSVYWKGWFGARV
TNSSVWVEP.

Variant Fbs1-8
(SEQ ID NO: 8)
MGSSHHHHHHHHSSGENLYFQSHMASQQEGLVPEGSADEERDHWQQFY
FLSKRRRNLLRNPCGEEDLEGWSDVEHGGDGWRVRRLPGDGGVEFTQD
DSVKKYFASSYRWCRKAQVIDLQAEGYWEELLDTTQPAIVVKDWYSGR
TDAGSLYELTVRLLSENEDVLAEFATGQVAVPEDGSWMEISHTFIDYG
PGVRFVRFEHGGQDSVYWKGWFGARVTNSSVWVEP.

The Fbs1-8 variant without the N-terminal immobilization tag has the following sequence:

(SEQ ID NO: 18)
QQEGLVPEGSADEERDHWQQFYFLSKRRRNLLRNPCGEEDLEGWSDVE
HGGDGWRVRRLPGDGGVEFTQDDSVKKYFASSYRWCRKAQVIDLQAEG
YWEELLDTTQPAIVVKDWYSGRTDAGSLYELTVRLLSENEDVLAEFAT
GQVAVPEDGSWMEISHTFIDYGPGVRFVRFEHGGQDSVYWKGWFGARV
TNSSVWVEP.

### Example 11

### Variants Fbs1-4 and Fbs-5

The Fbs1-4 and Fbs1-5 variants are Fbs1-1 with alanine substitutions at positions 214 and 216, respectfully, to reduce affinity (so glycoproteins elute more easily from an FBS1 affinity column). However, this significantly reduced binding by BLI (see Example 12 and FIG. 11). The sequences of Fbs1-4 and Fbs1-5 are shown below:

Variant Fbs1-4
(SEQ ID NO: 4)
MGSSHHHHHHHHSSGENLYFQSHMASQQEGLVPEGSADEERDHWQQFY
FLSKRRRNLLRNPCGEEDLEGWSDVEHGGDGWRVEELPGDNGVEFTQD
DSVKKYFASSFEWCRKAQVIDLQAEGYWEELLDTTQPAIVVKDWYSGA
TDAGSLYELTVRLLSENEDVLAEFATGQVAVPEDGSWMEISHTFIDYG
PGVRFVRFEHGGQDSVYWKGWFGARVTNSSVWVEP

The Fbs1-4 variant without the N-terminal immobilization tag has the following sequence:

(SEQ ID NO: 14)
QQEGLVPEGSADEERDHWQQFYFLSKRRRNLLRNPCGEEDLEGWSDVE
HGGDGWRVEELPGDNGVEFTQDDSVKKYFASSFEWCRKAQVIDLQAEG
YWEELLDTTQPAIVVKDWYSGATDAGSLYELTVRLLSENEDVLAEFAT
GQVAVPEDGSWMEISHTFIDYGPGVRFVRFEHGGQDSVYWKGWFGARV
TNSSVWVEP.

Variant Fbs1-5
(SEQ ID NO: 5)
MGSSHHHHHHHHSSGENLYFQSHMASQQEGLVPEGSADEERDHWQQFY
FLSKRRRNLLRNPCGEEDLEGWSDVEHGGDGWRVEELPGDNGVEFTQD
DSVKKYFASSFEWCRKAQVIDLQAEGYWEELLDTTQPAIVVKDWYSGR
TAAGSLYELTVRLLSENEDVLAEFATGQVAVPEDGSWMEISHTFIDYG
PGVRFVRFEHGGQDSVYWKGWFGARVTNSSVWVEP.

The Fbs1-5 variant without the N-terminal immobilization tag has the following sequence:

(SEQ ID NO: 15)
QQEGLVPEGSADEERDHWQQFYFLSKRRRNLLRNPCGEEDLEGWSDVE
HGGDGWRVEELPGDNGVEFTQDDSVKKYFASSFEWCRKAQVIDLQAEG
YWEELLDTTQPAIVVKDWYSGRTAAGSLYELTVRLLSENEDVLAEFAT
GQVAVPEDGSWMEISHTFIDYGPGVRFVRFEHGGQDSVYWKGWFGARV
TNSSVWVEP.

### Example 12

### Binding Specificity of Fbs Variants Using Biolayer Interferometry

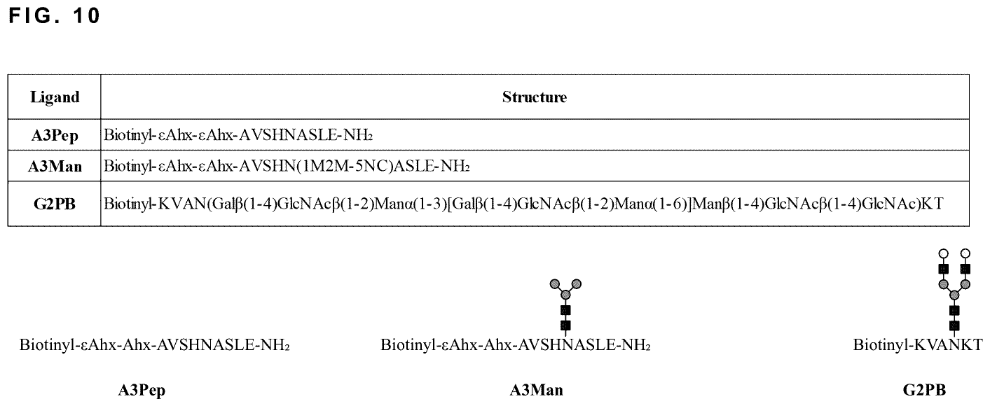

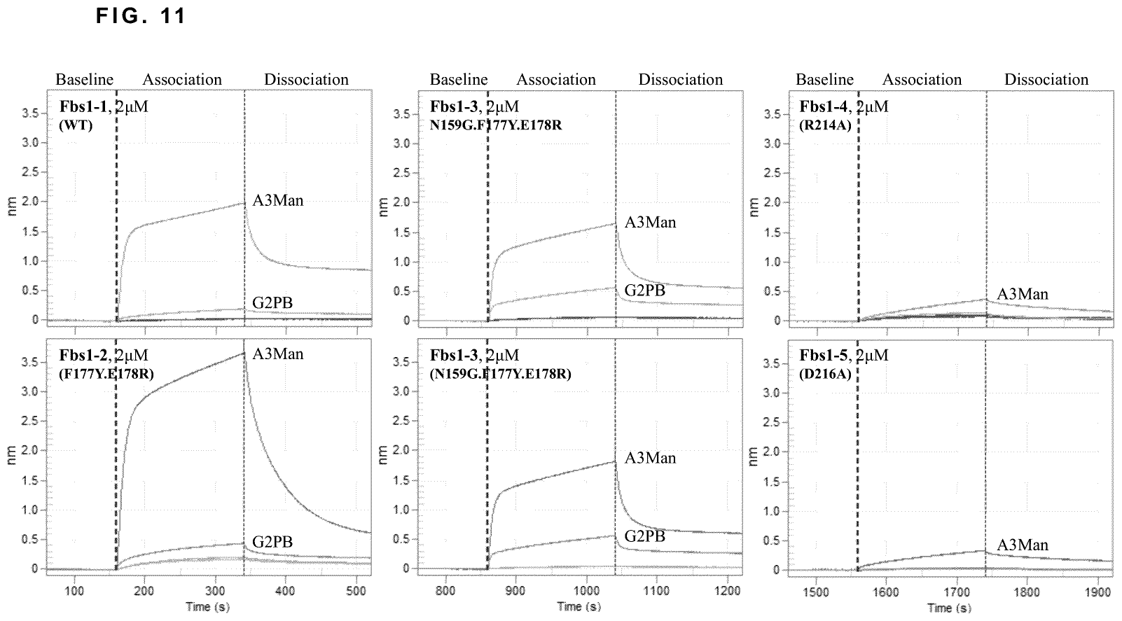

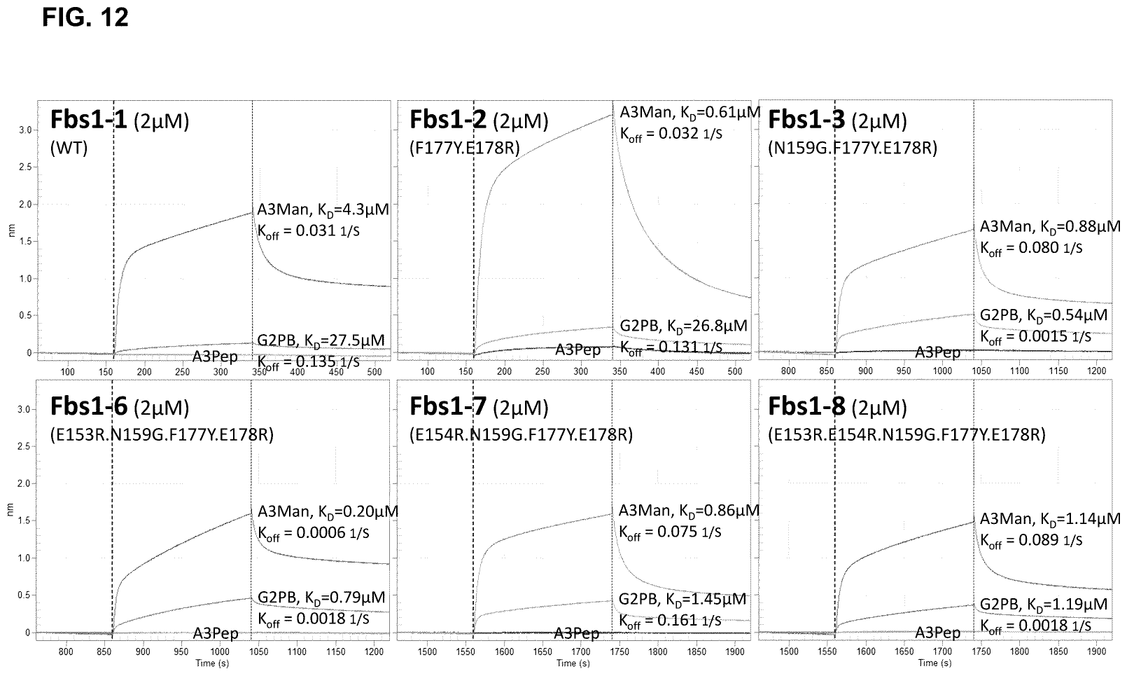

Using Biolayer Interferometry (BLI) methods described in more detail in Example 2, the specificity of Fbs variants Fbs1-3, Fbs1-4, Fbs1-5, Fbs1-6, Fbs1-7, and Fbs1-8 was measured by binding to biotinylated ligands immobilized on SAX biosensors. The structure of the various ligands is shown in FIG. 10. The results are shown in FIG. 11 and FIG. 12. FIG. 11 shows that Fbs1-3 binds best to G2PB.

### Example 13

### Western Blotting of Fbs1 Variants

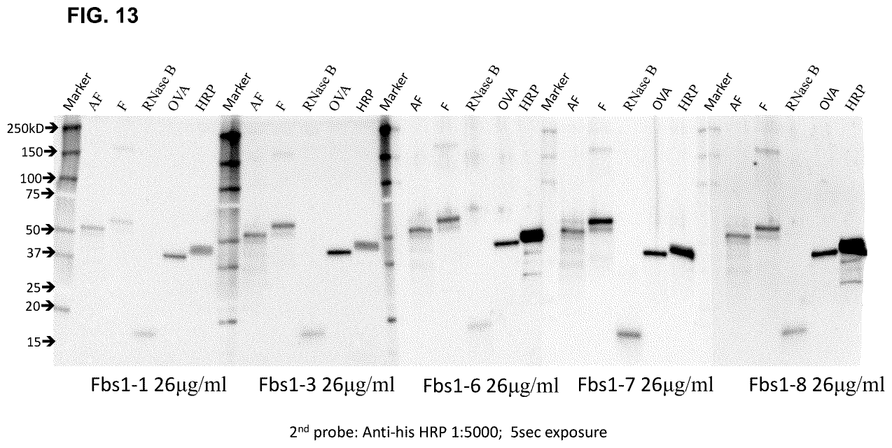

Using western blotting methods described in more detail in Example 3, samples of glycoproteins (Asialofetuin, Fetuin, RNase B, Ovalbumin, and Horse Radish Peroxidase) were separated by SDS-PAGE, blotted on nitrocellulose membrane, and probed by Fbs1-2, Fbs1-3, Fbs1-6, Fbs1-7, or Fbs1-8. The results shown in FIG. 13. The results indicate that all the mutants are promising, with variant Fbs1-7 likely the best.

### Example 14

### Glycan Array Screening of Fbs1-7

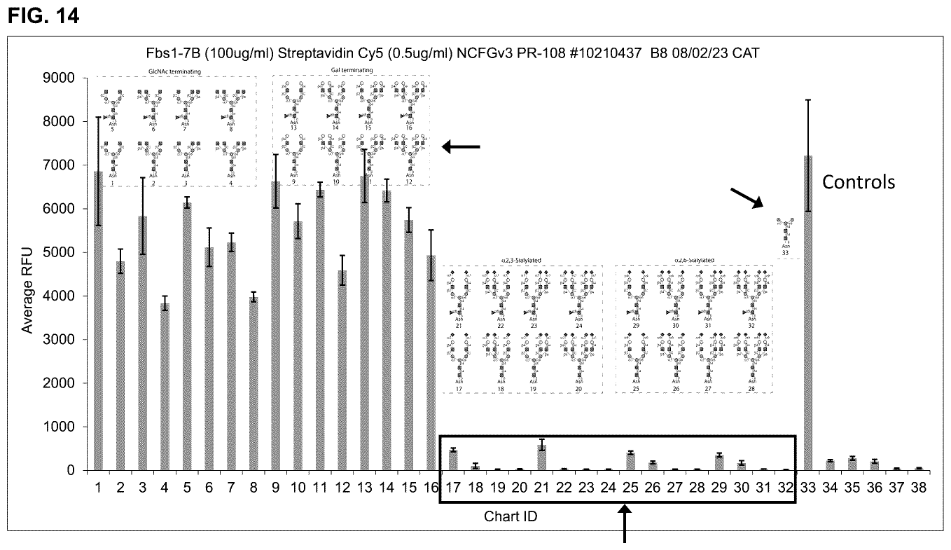

Following methods described in more detail in Example 5, an array of immobilized glycans (structures from Gao et al., 2019, Cell Chem Biol; 26(4): 535-547) was incubated with 100 μg/mL biotinylated Fbs1-7, followed by 0.5 μg/mL of a streptavidin Cy5-635 conjugate. The results are shown in FIG. 14. Fbs1-7 binds most N-glycans better than Fbs1-1 (FIG. 5), except for sialylated N-glycans.

### Example 15

### Additional Mouse Fbs1 Variants for N-Glycan Detection Reagents

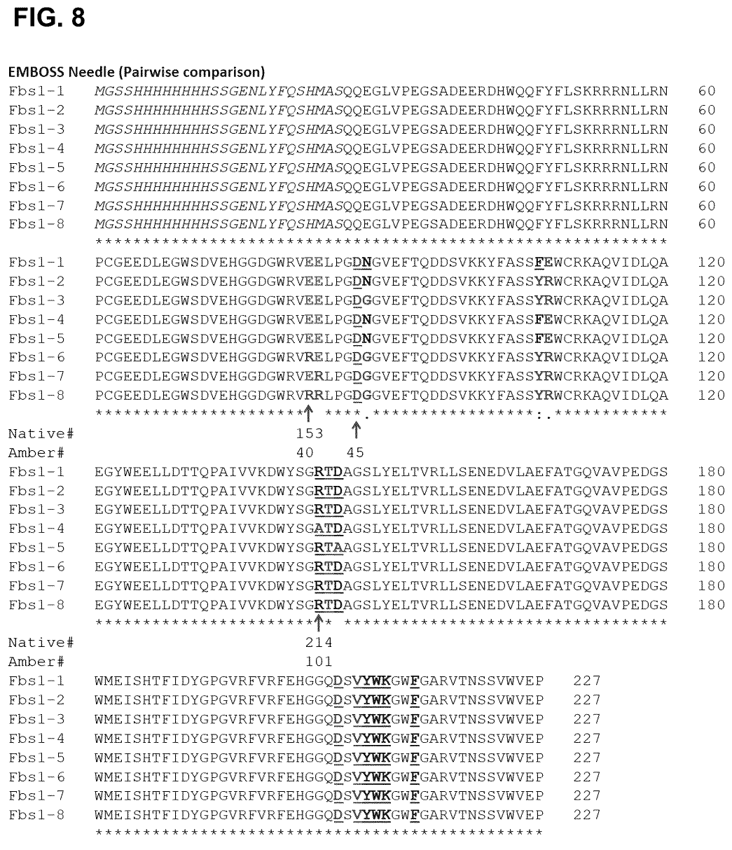

As described in the previous examples, native mouse Fbs1 (mmFBXO2 (SEQ ID NO: 9)), variant Fbs1-1 (SEQ ID NO: 1), variant Fbs1-2 (SEQ ID NO: 2), variant Fbs1-3 (SEQ ID NO: 3), variant Fbs1-4 (SEQ ID NO: 4), variant Fbs1-5 (SEQ ID NO: 5), variant Fbs1-6 (SEQ ID NO: 6), variant Fbs1-7 (SEQ ID NO: 1), and variant Fbs1-8 (SEQ ID NO: 8) were used for targeted modifications, computational modeling, and directed evolution to develop proteins that are able to specifically bind to N-glycosylated peptides and proteins, and do not show substantial binding to non-glycosylated peptides and proteins, and are suitable for use as N-glycan detection reagents. The amino acid sequences of these variants are shown in FIG. 8.

Parameters such as signal strength, or affinity for different ligands will be addressed to develop additional mouse Fbs1-derived variants with properties related to, inter alia, binding affinity, binding kinetics, and/or binding specificities that could be focused to develop desired properties of specific utility in one or more application such as affinity chromatography, flow cytometry, immunohistochemistry, Western blot analysis, purification, sample enrichment, or for therapeutic and/or diagnostic applications.

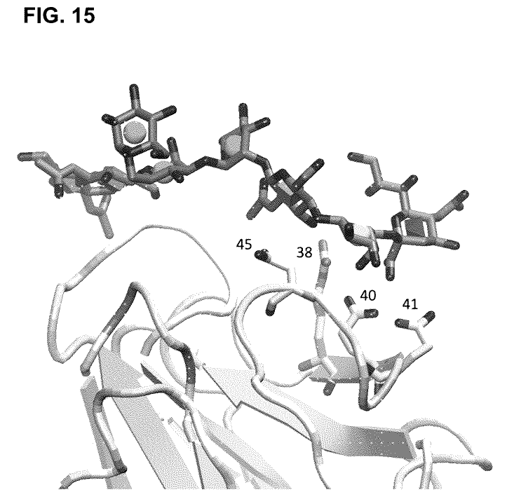

FIG. 15 shows the last frame from MD simulation of sialylated glycans with wild type Fbs1. As used herein, the term wildtype (wt) refers to a protein encoded by a gene that has a sequence of a gene as it naturally occurs in an organism, and that has not been altered by human intervention. For structure preparation for MD, coordinates for the sugar-binding domain of murine Fbs1(117-297) were taken from the wild-type crystal structure (PDBID: 1UMI). The crystallographic chitobiose ligand was replaced by the sialylated octasaccharide generated using GLYCAM-Web. Counter ions (Na+) were added as necessary to neutralize the protein. Crystallographic waters and non-standard residues were deleted, hydrogen atoms were added, and the co-complex solvated with TIP3P waters. For simulation details, the solvated co-complex was energy minimized, heated to 300 K and equilibrated. A production data set of 100 ns was collected, and snapshots analyzed at 10 ps intervals (10,000 snapshots) using MM-GBSA with IGB=2. Results are shown in Tables 1 and 2 below.

This analysis focused on the interactions between the native Fbs1 protein and the terminal Neu5Ac residue in the sialylated octasaccharide Neu5Aca2-6Galb1-4G1cNAcb1-2Mana1-3[Mana1-6]Manb1-4GlcNAcb1-4GlcNAcOH. This glycan contains only one sialic acid, which is attached to the Manal-3 arm. Modeling led to the following conclusions (AMBER numbering, FIG. 15):

ARG 38(151) stabilizes the interaction between native Fbs1 and the Neu5Ac residue.

However, GLU 40(153) and GLU 41(154) significantly destabilize this interaction.

Overall, the Neu5Ac and Gal residues make unfavorable interactions with the protein, although the net interaction with the glycan is still favorable.

No tepid residues were identified that were proximal to the Neu5Ac residue, although ASP 45(158) could be considered tepid (ΔΔG=−0.52 kcal/mol).

ASP 45(158) is proximal to the Gal residue.

The glycosidic linkage between the galactose and the Neu5Ac residue was markedly flexible, populating several conformations, which is consistent with the lack of a strong interaction between the Neu5Ac and the protein.

In view of these conclusions:

GLU 40(153) and GLU41(154) will be mutated to positively-charged residues (Arg, Lys), or to polar neutral residues (Gln, Asn, His).

In addition to the Glu to Arg mutations of variants Fbs1-6, Fbs1-7, and Fbs1-8, these residues may be mutated to Lys and polar neutral residues.

Variants to randomize position 45(158) will be produced.

TABLE 1
Protein Per-residue Average Interaction Energies^{a}

| | | | | | | | |
|---|---|---|---|---|---|---|---|
| | | Native | | | Polar | Non-polar | |
| Residue | Amber # | FBS1 # | ΔE_{VdW} | ΔE_{Elec} | Solvation | Solvation | ΔG_{Binding} |

Favorable residues

| | | | | | | | |
|---|---|---|---|---|---|---|---|
| TRP | 167 | 280 | −7.55 | −3.30 | 3.84 | −0.72 | −7.74 |
| LYS | 168 | 281 | −2.24 | −17.37 | 16.15 | −0.37 | −3.82 |
| TYR | 166 | 279 | −2.20 | −3.05 | 3.25 | −0.34 | −2.33 |
| PHE | 171 | 284 | −2.09 | −0.16 | 0.26 | −0.35 | −2.33 |
| ARG | 38 | 151 | −0.56 | −22.79 | 22.17 | −0.22 | −1.40 |
| TYR | 64 | 177 | −1.00 | −0.36 | 0.39 | −0.05 | −1.03 |
| ASP | 103 | 216 | −1.06 | 1.27 | −0.75 | −0.27 | −0.81 |
| PRO | 43 | 156 | −0.60 | −1.39 | 1.36 | −0.17 | −0.81 |
| GLY | 44 | 157 | −0.70 | −1.17 | 1.28 | −0.10 | −0.70 |
| ASP | 45 | 158 | −2.56 | 6.24 | −3.72 | −0.49 | −0.52 |

Unfavorable residues

| | | | | | | | |
|---|---|---|---|---|---|---|---|
| GLU | 40 | 153 | −0.38 | 16.84 | −15.02 | −0.17 | 1.27 |
| GLU | 41 | 154 | −0.81 | 20.58 | −18.40 | −0.21 | 1.16 |

TABLE 2
Ligand Per-residue Average Interaction Energies

| | | | Polar | Non-polar | |
|---|---|---|---|---|---|
| Residue | ΔE_{VdW} | ΔE_{Elec} | Solvation | Solvation | ΔG_{Binding} |
| GlcNAc1 | −7.41 | 7.42 | −20.92 | −7.23 | −6.68 |
| GlcNAc2 | −5.62 | −3.53 | 7.03 | −0.64 | −2.76 |
| Manb1-4 | −2.64 | 15.52 | −14.22 | −0.38 | −1.72 |
| Mana1-6 | −1.45 | −21.38 | 23.26 | −0.23 | 0.20 |
| Mana1-3 | −0.81 | −0.47 | 1.75 | −0.06 | 0.40 |
| GlcNAc1-4 | −2.89 | −5.02 | 6.31 | −0.53 | −2.13 |
| Galb1-4 | −2.12 | −4.60 | 8.51 | −0.39 | 1.41 |
| Neu5Aca2-6 | −2.20 | 74.15 | −68.21 | −0.70 | 3.04 |

^{a}Energies are in kcal/mol

### Example 16

### Fbs2 and Fbs3 Variants for N-Glycan Detection Reagents

Mouse (Mus musculus) Fbs proteins Fbs2 (mmFBXO6) and Fbs3 (FBXO27) will be used for targeted modifications, computational modeling and directed evolution to develop variants that are able to specifically bind to N-glycosylated peptides and proteins, do not show substantial binding to non-glycosylated peptides and proteins, and are suitable for use as N-glycan detection reagents. Differences between mouse Fbs2 and/or mouse Fbs2-derived variants, and/or mouse Fbs3 and/or mouse Fbs3-derived variants will be observed for parameters such as signal strength, or affinity for different ligands, which indicate potential for tunable affinities and binding kinetics, and tunable specificities that are addressed to develop desired properties of specific utility in one or more applications. The native mouse F-box protein sequences for Fbs2 (mmFBXO6 (SEQ ID NO: 21)) and Fbs3 (FBXO27 (SEQ ID NO: 22)) are provided below.

Fbs2 (mmFBXO6)
(SEQ ID NO: 21)
MVHINELPENILLELFIHIPAPQLLRNCRLVCRLWRDLIDVVSLWKRK
SLREGFFTKDRCEPVEDWKVFYILCSLQRNLLRNPCAEENLSSWRIDS
NGGDRWKVETLPGSCGTSFPDNKVKKYFVTSFEMCLKSQMVDLKAEGY
CEELMDTFRPDIVVKDWVAPRADCGCTYQLRVQLASADYIVLASFEPP
PVTFQQWNDAKWQEISHTFSDYPPGVRHILFQHGGQDTQFWKGWYGPR
VTNSSIIISHRTAKNPPPARTLPEETVVIGRRRRASDSNTHEGFFWQG
LWQRLRR
Fbs3 (FBXO27)
(SEQ ID NO: 22)
MGAWISRTRVPTPEPDPQEVLDLSRLPPELLLLVLSHVPPRTLLMHCR
RVCRAWRALVDGQALWLLLLARDHSAAGRALLTLARRCLPPAHEDTPC
PLGQFCALRPLGRNLISNPCGQEGLRKWMVRHGGDGWVVEKNRKPVPG
APSQTCFVTSFSWCRKKQVVDLVEKGLWPELLDSGGVEIAVSDWWGAR
HDSGCKYRLFVTLLDAHQNVIDKFSAVPDPIEQWNNDIYLQVTHVFSG
IRRGIRFVSFEHWGQDTQFWAGHYGARVTNSSVIIRVCQS

FIG. 1B shows a multiple sequence alignment of Fbs1 (mmFBXO2), Fbs2 (mmFBXO6) and Fbs3 (mmFBXO27) using the CLUSTAL Omega (1.2.4) multiple sequence alignment tool (available on the worldwide at, for example, clustal.org/omega/).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Sequence Listing Free Text

| | |
|---|---|
| SEQ ID NO: 1 | amino acid sequence of the Fbs1-1 variant with N-terminal non-Fbs-1 tag |
| SEQ ID NO: 2 | amino acid sequence of the Fbs1-2 variant with N-terminal non-Fbs1 tag |
| SEQ ID NO: 3 | amino acid sequence of the Fbs1-3 variant with N-terminal non-Fbs-1 tag |
| SEQ ID NO: 4 | amino acid sequence of the Fbs1-4 variant with N-terminal non-Fbs1 tag |
| SEQ ID NO: 5 | amino acid sequence of the Fbs1-5 variant with N-terminal non-Fbs-1 tag |
| SEQ ID NO: 6 | amino acid sequence of the Fbs1-6 variant with N-terminal non-Fbs1 tag |
| SEQ ID NO: 7 | amino acid sequence of the Fbs1-7 variant with N-terminal non-Fbs-1 tag |
| SEQ ID NO: 8 | amino acid sequence of the Fbs1-8 variant with N-terminal non-Fbs1 tag |
| SEQ ID NO: 9 | wild-type mouse (Mus musculus) mmFBXO2 amino acid sequence |
| SEQ ID NO: 10 | generic amino acid sequence sugar binding domain of Fbs1 |
| | variants/truncated with Xaa's at wt positions |
| | 153/154/158/159/160/177/178/214/216 and no N-terminal non-Fbs1 |
| | immobilization tag |
| SEQ ID NO: 11 | amino acid sequence of the Fbs1-1 variant without N-terminal tag |
| SEQ ID NO: 12 | amino acid sequence of the Fbs1-2 variant without N-terminal tag |
| SEQ ID NO: 13 | amino acid sequence of the Fbs1-3 variant without N-terminal tag |
| SEQ ID NO: 14 | amino acid sequence of the Fbs1-4 variant without N-terminal tag |
| SEQ ID NO: 15 | amino acid sequence of the Fbs1-5 variant without N-terminal tag |
| SEQ ID NO: 16 | amino acid sequence of the Fbs1-6 variant without N-terminal tag |
| SEQ ID NO: 17 | amino acid sequence of the Fbs1-7 variant without N-terminal tag |
| SEQ ID NO: 18 | amino acid sequence of the Fbs1-8 variant without N-terminal tag |
| SEQ ID NO: 19 | amino acid sequence of the N-terminal non-Fbs-1 immobilization tag |
| SEQ ID NO: 20 | human (Homo sapiens) F-Box only protein 2 (hsFBXO2) amino acid |
| | sequence |
| SEQ ID NO: 21 | amino acid sequence of native mouse (Mus musculus) Fbs2 F-box protein |
| | (mmFBXO6) |
| SEQ ID NO: 22 | amino acid sequence of native mouse (Mus musculus) Fbs3 F-box protein |
| | (FBXO27) |

What is claimed is:

1. A binding reagent that specifically binds to N-glycosylated peptides and proteins and does not show substantial binding to non-glycosylated peptides and proteins, wherein the binding reagent comprises an amino acid sequence having at least 90% identity to amino acid residues 125 to 297 (the sugar binding domain) of the murine Fbs1 protein (SEQ ID NO: 9) and having an amino acid substitution at position 153 and/or 154 of SEQ ID NO:9.

2. The binding reagent of claim 1 further comprising an amino acid sequence having at least 90% sequence identity to amino acid residues 96 to 124 (the linker domain) of the murine Fbs1 protein (SEQ ID NO: 9).

3. The binding reagent of claim 1 further comprising a N-terminal immobilization tag.

4. The binding reagent of claim 3 wherein the N-terminal immobilization tag comprises the amino acid sequence MGSSHHHHHHHHSSGENLYFQSHMAS (SEQ ID NO: 19).

5. The binding reagent of claim 1, wherein the substitution at position 153 and/or 154 comprises a E153R and/or a E154R substitution.

6. The binding reagent of claim 1 further comprising an amino acid substitution at position 177 and/or 178.

7. The binding reagent of claim 6 wherein the substitution at position 177 and/or 178 comprises a F177Y and/or a E178R substitution.

8. The binding reagent protein of claim 1 further an comprising amino acid substitution at position 158 and/or 159.

9. The binding reagent of claim 6 further comprising an amino acid substitution at position 158 and/or 159.

10. The binding reagent of claim 9 wherein the substitution at position 159 comprises a N159G substitution.

11. The binding reagent of claim 10, wherein the binding reagent comprises at least 90% sequence identity to SEQ ID NO: 6, SEQ ID NO: 7, SEQ ID NO: 8, SEQ ID NO: 16, SEQ ID NO: 17, or SEQ ID NO: 18.

12. A composition comprising the binding reagent of claim 1.

13. A conjugate comprising a first component comprising a binding reagent of claim 1 covalently linked to a second component.

14. An affinity matrix comprising the binding reagent of claim 1.

15. The affinity matrix of claim 14 selected from the group consisting of a solid support, surface, a column, a resin, a bead, a particle, and a nanoparticle.

16. An isolated polynucleotide encoding the binding reagent of claim 1.

17. A vector or host cell comprising the polynucleotide of claim 16.

18. A method for detecting an N-glycosylated protein in a sample, the method comprising contacting the sample with the binding reagent of claim 1 under conditions to allow binding of the binding reagent to the N-glycosylated protein and detecting the N-glycosylated protein bound to the binding reagent.

19. A method for enriching, isolating, or purifying an N-glycosylated protein, the method comprising contacting a biological or laboratory sample with the binding reagent of claim 1 under conditions to allow binding of the binding reagent to an N-glycosylated protein in the sample, and recovering the N-glycosylated protein bound to the binding reagent.

20. The method of claim 19, further comprising separating the recovered N-glycosylated protein from the binding reagent.


SEQUENCE LISTING (22 sequences)

SEQ ID NO: 1 - protein, 227 aa, synthetic construct
MGSSHHHHHHHHSSGENLYFQSHMASQQEGLVPEGSADEERDHWQQFYFLSKRRRNLLRNPCGEEDLEGWSDVEHGGDGWRVEELPGDNGVEFTQDDSVKKYFASSFEWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 2 - protein, 227 aa, synthetic construct
MGSSHHHHHHHHSSGENLYFQSHMASQQEGLVPEGSADEERDHWQQFYFLSKRRRNLLRNPCGEEDLEGWSDVEHGGDGWRVEELPGDNGVEFTQDDSVKKYFASSYRWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 3 - protein, 227 aa, synthetic construct
MGSSHHHHHHHHSSGENLYFQSHMASQQEGLVPEGSADEERDHWQQFYFLSKRRRNLLRNPCGEEDLEGWSDVEHGGDGWRVEELPGDGGVEFTQDDSVKKYFASSYRWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 4 - protein, 227 aa, synthetic construct
MGSSHHHHHHHHSSGENLYFQSHMASQQEGLVPEGSADEERDHWQQFYFLSKRRRNLLRNPCGEEDLEGWSDVEHGGDGWRVEELPGDNGVEFTQDDSVKKYFASSFEWCRKAQVIDLQAEGYWEELLDTTQPAIVVKDWYSGATDAGSLYELTVRLLSENEDVLAEFATGQVAVPEDGSWMEISHTFIDYGPGVRFVRFEHGGQDSVYWKGWFGARVTNSSVWVEP

SEQ ID NO: 5 - protein, 227 aa, synthetic construct
MGSSHHHHHHHHSSGENLYFQSHMASQQEGLVPEGSADEERDHWQQFYFLSKRRRNLLRNPCGEEDLEGWSDVEHGGDGWRVEELPGDNGVEFTQDDSVKKYFASSFEWCRKAQVIDLQAEGYWEELLDTTQPAIVVKDWYSGRTAAGSLYELTVRLLSENEDVLAEFATGQVAVPEDGSWMEISHTFIDYGPGVRFVRFEHGGQDSVYWKGWFGARVTNSSVWVEP

SEQ ID NO: 6 - protein, 227 aa, synthetic construct
MGSSHHHHHHHHSSGENLYFQSHMASQQEGLVPEGSADEERDHWQQFYFLSKRRRNLLRNPCGEEDLEGWSDVEHGGDGWRVRELPGDGGVEFTQDDSVKKYFASSYRWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 7 - protein, 227 aa, synthetic construct
MGSSHHHHHHHHSSGENLYFQSHMASQQEGLVPEGSADEERDHWQQFYFLSKRRRNLLRNPCGEEDLEGWSDVEHGGDGWRVERLPGDGGVEFTQDDSVKKYFASSYRWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 8 - protein, 227 aa, synthetic construct
MGSSHHHHHHHHSSGENLYFQSHMASQQEGLVPEGSADEERDHWQQFYFLSKRRRNLLRNPCGEEDLEGWSDVEHGGDGWRVRRLPGDGGVEFTQDDSVKKYFASSYRWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 9 - protein, 297 aa, Mus musculus
MDGDGDPESVSHPEEASPEEQPEEAGAEASAEEEQLREAEEEEEAEAVEYLAELPEPLLLRVLAELPATELVQACRLVCLRWKELVDGAPLWLLKCQQEGLVPEGSADEERDHWQQFYFLSKRRRNLLRNPCGEEDLEGWSDVEHGGDGWRVEELPGDNGVEFTQDDSVKKYFASSFEWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 10 - protein, 172 aa, synthetic construct
NLLRNPCGEEDLEGWSDVEHGGDGWRVXXLPGXXGVEFTQDDSVKKYFASSXXWCRKAQVIDLQAEGYWEELLDTTQPAIVVKDWYSGXTXAGSLYELTVRLLSENEDVLAEFATGQVAVPEDGSWMEISHTFIDYGPGVRFVRFEHGGQDSVYWKGWFGARVTNSSVWVEP

SEQ ID NO: 11 - protein, 201 aa, synthetic construct
QQEGLVPEGSADEERDHWQQFYFLSKRRRNLLRNPCGEEDLEGWSDVEHGGDGWRVEELPGDNGVEFTQDDSVKKYFASSFEWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 12 - protein, 201 aa, synthetic construct
QQEGLVPEGSADEERDHWQQFYFLSKRRRNLLRNPCGEEDLEGWSDVEHGGDGWRVEELPGDNGVEFTQDDSVKKYFASSYRWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 13 - protein, 201 aa, synthetic construct
QQEGLVPEGSADEERDHWQQFYFLSKRRRNLLRNPCGEEDLEGWSDVEHGGDGWRVEELPGDGGVEFTQDDSVKKYFASSYRWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 14 - protein, 201 aa, synthetic construct
QQEGLVPEGSADEERDHWQQFYFLSKRRRNLLRNPCGEEDLEGWSDVEHGGDGWRVEELPGDNGVEFTQDDSVKKYFASSFEWCRKAQVIDLQAEGYWEELLDTTQPAIVVKDWYSGATDAGSLYELTVRLLSENEDVLAEFATGQVAVPEDGSWMEISHTFIDYGPGVRFVRFEHGGQDSVYWKGWFGARVTNSSVWVEP

SEQ ID NO: 15 - protein, 201 aa, synthetic construct
QQEGLVPEGSADEERDHWQQFYFLSKRRRNLLRNPCGEEDLEGWSDVEHGGDGWRVEELPGDNGVEFTQDDSVKKYFASSFEWCRKAQVIDLQAEGYWEELLDTTQPAIVVKDWYSGRTAAGSLYELTVRLLSENEDVLAEFATGQVAVPEDGSWMEISHTFIDYGPGVRFVRFEHGGQDSVYWKGWFGARVTNSSVWVEP

SEQ ID NO: 16 - protein, 201 aa, synthetic construct
QQEGLVPEGSADEERDHWQQFYFLSKRRRNLLRNPCGEEDLEGWSDVEHGGDGWRVRELPGDGGVEFTQDDSVKKYFASSYRWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 17 - protein, 201 aa, synthetic construct
QQEGLVPEGSADEERDHWQQFYFLSKRRRNLLRNPCGEEDLEGWSDVEHGGDGWRVERLPGDGGVEFTQDDSVKKYFASSYRWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 18 - protein, 201 aa, synthetic construct
QQEGLVPEGSADEERDHWQQFYFLSKRRRNLLRNPCGEEDLEGWSDVEHGGDGWRVRRLPGDGGVEFTQDDSVKKYFASSYRWCRKAQVIDLQAEGYWEELLDTTQPAIVVKDWYSGRTDAGSLYELTVRLLSENEDVLAEFATGQVAVPEDGSWMEISHTFIDYGPGVRFVRFEHGGQDSVYWKGWFGARVTNSSVWVEP

SEQ ID NO: 19 - protein, 26 aa, synthetic construct
MGSSHHHHHHHHSSGENLYFQSHMAS

SEQ ID NO: 20 - protein, 296 aa, Homo sapiens
MDGDGDPESVGQPEEASPEEQPEEASAEEERPEDQQEEEAAAAAAYLDELPEPLLLRVLAALPAAELVQACRLVCLRWKELVDGAPLWLLKCQQEGLVPEGGVEEERDHWQQFYFLSKRRRNLLRNPCGEEDLEGWCDVEHGGDGWRVEELPGDSGVEFTHDESVKKYFASSFEWCRKAQVIDLQAEGYWEELLDTTQPAIVVKDWYSGRSDAGCLYELTVKLLSEHENVLAEFSSGQVAVPQDSDGGGWMEISHTFTDYGPGVRFVRFEHGGQDSVYWKGWFGARVTNSSVWVEP

SEQ ID NO: 21 - protein, 295 aa, Mus musculus
MVHINELPENILLELFIHIPAPQLLRNCRLVCRLWRDLIDVVSLWKRKSLREGFFTKDRCEPVEDWKVFYILCSLQRNLLRNPCAEENLSSWRIDSNGGDRWKVETLPGSCGTSFPDNKVKKYFVTSFEMCLKSQMVDLKAEGYCEELMDTFRPDIVVKDWVAPRADCGCTYQLRVQLASADYIVLASFEPPPVTFQQWNDAKWQEISHTFSDYPPGVRHILFQHGGQDTQFWKGWYGPRVTNSSIIISHRTAKNPPPARTLPEETVVIGRRRRASDSNTHEGFFWQGLWQRLRR

SEQ ID NO: 22 - protein, 280 aa, Mus musculus
MGAWISRTRVPTPEPDPQEVLDLSRLPPELLLLVLSHVPPRTLLMHCRRVCRAWRALVDGQALWLLLLARDHSAAGRALLTLARRCLPPAHEDTPCPLGQFCALRPLGRNLISNPCGQEGLRKWMVRHGGDGWVVEKNRKPVPGAPSQTCFVTSFSWCRKKQVVDLVEKGLWPELLDSGGVEIAVSDWWGARHDSGCKYRLFVTLLDAHQNVIDKFSAVPDPIEQWNNDIYLQVTHVFSGIRRGIRFVSFEHWGQDTQFWAGHYGARVTNSSVIIRVCQS